SEQUENCE LISTING (52 sequences)

SEQ ID NO: 1 - protein, 5 aa, synthetic construct
DTYMH

SEQ ID NO: 2 - protein, 17 aa, synthetic construct
RIDPANGNSKYVPKFQG

SEQ ID NO: 3 - protein, 12 aa, synthetic construct
FGYYVSDYAMAY

SEQ ID NO: 4 - protein, 121 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSS

SEQ ID NO: 5 - protein, 15 aa, synthetic construct
RAGESVDIFGVGFLH

SEQ ID NO: 6 - protein, 7 aa, synthetic construct
RASNLES

SEQ ID NO: 7 - protein, 9 aa, synthetic construct
QQTNEDPYT

SEQ ID NO: 8 - protein, 111 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIK

SEQ ID NO: 9 - protein, 121 aa, synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 10 - protein, 7 aa, synthetic construct
RASNRAT

SEQ ID NO: 11 - protein, 111 aa, synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 12 - protein, 17 aa, synthetic construct
RIDPANGNSKYADSVKG

SEQ ID NO: 13 - protein, 121 aa, synthetic construct
EVQLVESGGGLVQPGGSLRLSCAASGFNIKDTYMHWVRQAPGKGLEWVGRIDPANGNSKYADSVKGRFTISADTSKNTAYLQMNSLRAEDTAVYYCAPFGYYVSDYAMAYWGQGTTVTVSS

SEQ ID NO: 14 - protein, 7 aa, synthetic construct
RGSNRAT

SEQ ID NO: 15 - protein, 111 aa, synthetic construct
EIVLTQSPATLSLSPGERATLSCRAGESVDIFGVGFLHWYQQKPGQAPRLLIYRGSNRATGIPARFSGSGSGTDFTLTISSLEPEDFAVYYCQQTNEDPYTFGQGTKLEIK

SEQ ID NO: 16 - protein, 9 aa, synthetic construct
QQTYEYPYT

SEQ ID NO: 17 - protein, 111 aa, synthetic construct
EIVLTQSPATLSLSPGERATLSCRAGESVDIFGVGFLHWYQQKPGQAPRLLIYRGSNRATGIPARFSGSGSGTDFTLTISSLEPEDFAVYYCQQTYEYPYTFGQGTKLEIK

SEQ ID NO: 18 - protein, 9 aa, synthetic construct
QQTVEYPYT

SEQ ID NO: 19 - protein, 111 aa, synthetic construct
EIVLTQSPATLSLSPGERATLSCRAGESVDIFGVGFLHWYQQKPGQAPRLLIYRGSNRATGIPARFSGSGSGTDFTLTISSLEPEDFAVYYCQQTVEYPYTFGQGTKLEIK

SEQ ID NO: 20 - protein, 9 aa, synthetic construct
QQTSEYPYT

SEQ ID NO: 21 - protein, 111 aa, synthetic construct
EIVLTQSPATLSLSPGERATLSCRAGESVDIFGVGFLHWYQQKPGQAPRLLIYRGSNRATGIPARFSGSGSGTDFTLTISSLEPEDFAVYYCQQTSEYPYTFGQGTKLEIK

SEQ ID NO: 22 - protein, 9 aa, synthetic construct
QQTTEYPYT

SEQ ID NO: 23 - protein, 111 aa, synthetic construct
EIVLTQSPATLSLSPGERATLSCRAGESVDIFGVGFLHWYQQKPGQAPRLLIYRGSNRATGIPARFSGSGSGTDFTLTISSLEPEDFAVYYCQQTTEYPYTFGQGTKLEIK

SEQ ID NO: 24 - protein, 7 aa, synthetic construct
RGSNRMT

SEQ ID NO: 25 - protein, 111 aa, synthetic construct
EIVLTQSPATLSLSPGERATLSCRAGESVDIFGVGFLHWYQQKPGQAPRLLIYRGSNRMTGIPARFSGSGSGTDFTLTISSLEPEDFAVYYCQQTNEDPYTFGQGTKLEIK

SEQ ID NO: 26 - protein, 7 aa, synthetic construct
  VARIANT 2: note = X = A or G
  VARIANT 6: note = X = A or M
RXSNRXT

SEQ ID NO: 27 - protein, 7 aa, synthetic construct
  VARIANT 6: note = X = A or M
RGSNRXT

SEQ ID NO: 28 - protein, 9 aa, synthetic construct
  VARIANT 4: note = X = N, Y, V, S or T
  VARIANT 6: note = X = D or Y
QQTXEXPYT

SEQ ID NO: 29 - protein, 111 aa, synthetic construct
  VARIANT 55: note = X = A or G
  VARIANT 59: note = X = A or M
  VARIANT 96: note = X = N, Y, V, S or T
  VARIANT 98: note = X = D or Y
EIVLTQSPATLSLSPGERATLSCRAGESVDIFGVGFLHWYQQKPGQAPRLLIYRXSNRXTGIPARFSGSGSGTDFTLTISSLEPEDFAVYYCQQTXEXPYTFGQGTKLEIK

SEQ ID NO: 30 - protein, 111 aa, synthetic construct
  VARIANT 59: note = X = A or M
  VARIANT 96: note = X = N, Y, V, S or T
  VARIANT 98: note = X = D or Y
EIVLTQSPATLSLSPGERATLSCRAGESVDIFGVGFLHWYQQKPGQAPRLLIYRGSNRXTGIPARFSGSGSGTDFTLTISSLEPEDFAVYYCQQTXEXPYTFGQGTKLEIK

SEQ ID NO: 31 - protein, 30 aa, synthetic construct
EVQLVESGGGLVQPGGSLRLSCAASGFNIK

SEQ ID NO: 32 - protein, 14 aa, synthetic construct
WVRQAPGKGLEWVG

SEQ ID NO: 33 - protein, 32 aa, synthetic construct
RFTISADTSKNTAYLQMNSLRAEDTAVYYCAP

SEQ ID NO: 34 - protein, 11 aa, synthetic construct
WGQGTTVTVSS

SEQ ID NO: 35 - protein, 23 aa, synthetic construct
EIVLTQSPATLSLSPGERATLSC

SEQ ID NO: 36 - protein, 15 aa, synthetic construct
WYQQKPGQAPRLLIY

SEQ ID NO: 37 - protein, 32 aa, synthetic construct
GIPARFSGSGSGTDFTLTISSLEPEDFAVYYC

SEQ ID NO: 38 - protein, 10 aa, synthetic construct
FGQGTKLEIK

SEQ ID NO: 39 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 107 aa, Homo sapiens [sequence omitted]

SEQ ID NO: 41 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 43 - protein, 15 aa, synthetic construct
RAGESVDHFGVGFLH

SEQ ID NO: 44 - protein, 111 aa, synthetic construct
EIVLTQSPATLSLSPGERATLSCRAGESVDHFGVGFLHWYQQKPGQAPRLLIYRASNRATGIPARFSGSGSGTDFTLTISSLEPEDFAVYYCQQTNEDPYTFGQGTKLEIK

SEQ ID NO: 45 - protein, 5 aa, synthetic construct
SYAMN

SEQ ID NO: 46 - protein, 19 aa, synthetic construct
RIRSKYNNYATYYADSVKG

SEQ ID NO: 47 - protein, 14 aa, synthetic construct
ASNFPASYVSYFAY

SEQ ID NO: 48 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 49 - protein, 14 aa, synthetic construct
GSSTGAVTTSNYAN

SEQ ID NO: 50 - protein, 7 aa, synthetic construct
GTNKRAP

SEQ ID NO: 51 - protein, 9 aa, synthetic construct
ALWYSNLWV

SEQ ID NO: 52 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

(19) United States
(12) Patent Application Publication
(10) Pub. No.: US 2026/0071002 A1
(43) Pub. Date: Mar. 12, 2026

(54) ANTIBODIES BINDING TO CEACAM5

(71) Applicant: Hoffmann-La Roche Inc., Little Falls, NJ (US)

(72) Inventors: Alexander Christian BUJOTZEK, München (DE); Claudia FERRARA KOLLER, Zug (CH); Maximiliane KOENIG, Deisenhofen (DE); Ekkehard MÖESSNER, Kreuzlingen (CH); Diana Angela PIPPIG, Muenchen (DE); Johannes THEN, Herrsching (DE); Inja WALDHAUER, Zürich (CH); Wing Ki WONG, München (DE)

(73) Assignee: Hoffmann-La Roche Inc., Little Falls, NJ (US)

(21) Appl. No.: 18/985,979

(22) Filed: Dec. 18, 2024

(30) Foreign Application Priority Data
Dec. 20, 2023 (EP) 23218388.9

(51) Int. Cl. C07K 16/30; A61P 35/00; C07K 16/28

(52) U.S. Cl. CPC C07K 16/3007; A61P 35/00; C07K 16/2809; C07K 2317/24; C07K 2317/31; C07K 2317/52; C07K 2317/565; C07K 2317/92

(57) ABSTRACT

The present invention generally relates to antibodies that bind to CEACAM5. In addition, the present invention relates to polynucleotides encoding such antibodies, and vectors and host cells comprising such polynucleotides. The invention further relates to methods for producing the antibodies, and to methods of using them in the treatment of disease.

### CROSS-REFERENCE TO RELATED APPLICATIONS

This application claims priority under 35 U.S.C. 119 (b) to European Patent Application No. 23218388.9, filed Dec. 20, 2023, which is incorporated herein by reference in its entirety.

### SEQUENCE LISTING

The instant application contains a sequence listing which has been submitted electronically in .xml format and is hereby incorporated by reference in its entirety. Said.xml copy, created on Dec. 12, 2024, is named P38838-US_Sequence Listing.xml and is 52,810 bytes in size.

### FIELD OF THE INVENTION

The present invention generally relates to antibodies that bind to CEACAM5. In addition, the present invention relates to polynucleotides encoding such antibodies, and vectors and host cells comprising such polynucleotides. The invention further relates to methods for producing the antibodies, and to methods of using them in the treatment of disease.

### BACKGROUND

Carcinoembryonic antigen (CEA, also known as CEACAM5 or CD66e) is a glycoprotein having a molecular weight of about 180 kDa. CEACAM5 is a member of the immunoglobulin superfamily and contains seven domains that are linked to the cell membrane through a glycosylphosphatidylinositol (GPI) anchor (Thompson J. A., J Clin Lab Anal. 5:344-366, 1991). The seven domains include a single N-terminal Ig variable domain and six domains (A1-B1-A2-B2-A3-B3) homologous to the Ig constant domain (Hefta L J, et al., Cancer Res. 52:5647-5655, 1992).

The human CEA family contains 29 genes, of which 18 are expressed: 7 belonging to the CEA subgroup and 11 to the pregnancy-specific glycoprotein subgroup. Several CEA subgroup members are thought to possess cell adhesion properties. CEACAM5 is thought to have a role in innate immunity (Hammarström S., Semin Cancer Biol. 9 (2): 67-81 (1999)). Because of the existence of proteins closely related to CEACAM5, it can be challenging to raise anti-CEACAM5 antibodies that are specific for CEACAM5 with minimal cross-reactivity to the other closely related proteins.

CEACAM5 has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CECAM5A has been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9 (2-3): 145-53,1988; Nap et al., Cancer Res., 52 (8): 2329-23339,1992).

Tumors of epithelial origin, as well as their metastases, contain CEACAM5 as a tumor associated antigen. While the presence of CEACAM5 itself does not indicate transformation to a cancerous cell, the distribution of CEACAM5 is indicative. In normal tissue, CEACAM5 is generally expressed on the apical surface of the cell (Hammarström S., Semin Cancer Biol. 9 (2): 67-81 (1999)), making it inaccessible to antibody in the blood stream. In contrast to normal tissue, CEACAM5 tends to be expressed over the entire surface of cancerous cells (Hammarström S., Semin Cancer Biol. 9 (2): 67-81 (1999)). This change of expression pattern makes CEACAM5 accessible to antibody binding in cancerous cells. In addition, CEACAM5 expression increases in cancerous cells. Furthermore, increased CEACAM5 expression promotes increased intercellular adhesions, which may lead to metastasis (Marshall J., Semin Oncol., 30 (a Suppl. 8): 30-6, 2003).

As the prevalence of CEACAM5 expression is generally high in tumors but low in normal tissues, CEACAM5 is an attractive target antigen for cancer therapy. Accordingly, numerous antibodies have been raised against this target, one of which is the murine antibody T84.66 (Wagener et al., J Immunol 130, 2308 (1983), Neumaier et al., J Immunol 135, 3604 (1985)), which has also been chimerized (WO 1991/01990) and humanized (WO 2005/086875).

Further humanized variants of T84.66 with advantageous properties were subsequently made, in particular “humanized variant 1” (WO 2017/055389; SEQ ID NOs 22 and 23 therein; and SEQ ID NOs 9 and 11 herein). While having high affinity and specificity, as well as providing high acitivity in a T-cell bispecific antibody context (see WO 2017/055389), “humanized variant 1” (called “T84.66-LCHA herein) was found to have poor expression properties in common mammalian expression systems, representing a drawback for its use as therapeutic. There is therefore a need for a humanized anti-CEACAM5 antibody maintaining the favorable properties of “humanized variant 1” (T84.66-LCHA) but showing improved expression properties.

### SUMMARY OF THE INVENTION

The present invention provides novel antibodies, including multispecific (e.g. bispecific) antibodies, that bind CEACAM5 and have particularly favorable properties for production and therapeutic purposes. In particular, the antibodies can be well expressed, and also combine good produceability with low predicted immunogenicity, good binding affinity and specificity to CEACAM5 and good efficacy.

In one aspect, the invention provides an antibody that binds to CEACAM5, wherein the antibody comprises a heavy chain variable region (VH_{CEACAM5}) comprising the heavy chain complementarity determining region (HCDR) 1 of SEQ ID NO: 1, the HCDR 2 of SEQ ID NO: 12, and the HCDR 3 of SEQ ID NO: 3, and a light chain variable region (VL_{CEACAM5}) comprising the light chain complementarity determining region (LCDR) 1 of SEQ ID NO: 5, the LCDR 2 of SEQ ID NO: 26 and the LCDR 3 of SEQ ID NO: 28.

In some aspects,
(i) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 20;
(ii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 16;
(iii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 18;
(iv) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 22;
(v) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 7;
(vi) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 24, and the LCDR3 of SEQ ID NO: 7; or
(vii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 10, and the LCDR3 of SEQ ID NO: 7.

In some aspects, the VH_{CEACAM5} comprises an amino acid sequence that is at least about 95%, 96%, 97%, 98%, 99% or 100% identical to the amino acid sequence of SEQ ID NO: 13; and/or the VL_{CEACAM5} comprises an amino acid sequence that is at least about 95%, 96%, 97%, 98%, 99% or 100% identical to the amino acid sequence of SEQ ID NO: 29.

In some aspects,
(i) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 21;
(ii) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 17;
(iii) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 19;
(iv) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 23;
(v) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 15;
(vi) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 25; or
(vii) the VH_{CEACAM5} comprises an amino acid sequence that is at least about 95%, 96%, 97%, 98%, 99% or 100% identical to the amino acid sequence of SEQ ID NO: 13 and/or the VL_{CEACAM5} comprises an amino acid sequence that is at least about 95%, 96%, 97%, 98%, 99% or 100% identical to the amino acid sequence of SEQ ID NO: 11.

In one aspect, the invention provides an antibody that binds to CEACAM5, wherein the antibody comprises a heavy chain variable region (VH_{CEACAM5}) comprising an amino acid sequence that is at least about 95%, 96%, 97%, 98%, 99% or 100% identical to the amino acid sequence of SEQ ID NO: 13, and a light chain variable region (VL_{CEACAM5}) comprising an amino acid sequence that is at least about 95%, 96%, 97%, 98%, 99% or 100% identical to the amino acid sequence of SEQ ID NO: 29.

In one aspect, the invention provides an antibody that binds to CEACAM5, wherein the antibody comprises
(i) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 21;
(ii) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 17;
(iii) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 19;
(iv) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 23;
(v) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 15;
(vi) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 25; or
(vii) a VH_{CEACAM5} comprising an amino acid sequence that is at least about 95%, 96%, 97%, 98%, 99% or 100% identical to the amino acid sequence of SEQ ID NO: 13 and/or a VL_{CEACAM5} comprising an amino acid sequence that is at least about 95%, 96%, 97%, 98%, 99% or 100% identical to the amino acid sequence of SEQ ID NO: 11.

In some aspects, the antibody comprises an Fc region, particularly a human Fc region. In some aspects, the Fc region is an IgG, particularly an IgG₁, Fc region.

In some aspects, the antibody is a full-length antibody. In some aspects, the antibody is an IgG, particularly an IgG₁, antibody.

In some aspects, the antibody is an antibody fragment that binds to CEACAM5, particularly an antibody fragment selected from the group of an Fv molecule, a scFv molecule, a Fab molecule, and a F(ab′)₂ molecule.

In some aspects, the antibody is a multispecific, particularly a bispecific, antibody. In some aspects, the antibody is a bispecific antibody that binds to CEACAM5 and to CD3.

According to a further aspect of the invention there is provided an isolated polynucleotide encoding an antibody of the invention, and a host cell comprising the isolated polynucleotide of the invention.

In another aspect is provided a method of producing an antibody that binds to CEACAM5, comprising the steps of (a) culturing the host cell of the invention under conditions suitable for the expression of the antibody and optionally (b) recovering the antibody. The invention also encompasses an antibody that binds to CEACAM5 produced by the method of the invention.

The invention further provides a pharmaceutical composition comprising the antibody of the invention and a pharmaceutically acceptable carrier.

Also encompassed by the invention are methods of using the antibody and pharmaceutical composition of the invention. In one aspect, the invention provides an antibody or pharmaceutical composition according to the invention for use as a medicament. In one aspect is provided an antibody or pharmaceutical composition according to the invention for use in the treatment of a disease. In a specific aspect, the disease is cancer.

Also provided is the use of an antibody or pharmaceutical composition according to the invention in the manufacture of a medicament, and the use of an antibody or pharmaceutical composition according to the invention in the manufacture of a medicament for the treatment of a disease, particularly cancer. The invention also provides a method of treating a disease in an individual, comprising administering to said individual an effective amount of the antibody or pharmaceutical composition according to the invention. In a specific aspect, the disease is cancer.

### BRIEF DESCRIPTION OF THE DRAWINGS

FIG. 1 includes FIG. 1A and FIG. 1B, which are graphs illustrating the binding of T84.66 humanization variants to CEACAM5 expressing MKN-45 cells.

FIG. 2 includes FIG. 2A and FIG. 2B, which are graphs illustrating the binding of selected T84.66 humanization variants to CEACAM5 expressing LS180 cells.

FIG. 3 includes FIG. 3A and FIG. 3B, which are graphs illustrating the binding of optimized T84.66 humanization variants to CEACAM5 expressing MKN-45 cells.

FIG. 4 is a graph illustrating the binding of selected optimized T84.66 humanization variants to CEACAM5 expressing MKN-45 cells.

FIG. 5 is a graph illustrating the binding of selected optimized T84.66 humanization variants to CHO-CEACAM1 cells.

FIG. 6 is a graph illustrating the binding of selected optimized T84.66 humanization variants to CHO-CEACAM6 cells.

FIG. 7 includes FIGS. 7A-7E and are schematic illustrations of T-cell bispecific antibodies (TCBs). FIG. 7A illustrates the T-cell bispecific antibodies (TCBs) used in Example 6. The tested TCB antibody molecules were produced in a “2+1” format monovalently binding to CD3 and bivalently binding to CEACAM5, with a domain crossover (VH/VL exchange) in the CD3 binder, charge modifications in the CEACAM5 binders (EE=147E, 213E; RK=123R, 124K), and effector silencing mutations (P329G, L234A, L235A (“PG LALA”)) and knob-into-holes modifications (T366W/S354C (knob), T366S/L368A/Y407V/Y349C (hole)) in the Fc region. FIG. 7B shows components for the assembly of the TCB: light chain of anti-target antigen Fab molecule with charge modifications in CH1 and CL; FIG. 7C shows the light chain of anti-CD3 crossover Fabmolecule; FIG. 7D shows the heavy chain with knob and PG LALA mutations in Fc region FIG. 7E shows a heavy chain with hole and PG LALA mutations in the Fc region.

FIG. 8 shows a graph illustating Jurkat NFAT activation on LS180 tumor cells with parental CEACAM5 (T84.66-LCHA)-TCB and CEACAM5-TCB containing optimized CEACAM5 binder.

### DETAILED DESCRIPTION OF THE INVENTION

### I. Definitions

Terms are used herein as generally used in the art, unless otherwise defined in the following.

As used herein, the terms “first”, “second” or “third” with respect to antigen binding domains etc., are used for convenience of distinguishing when there is more than one of each type of moiety. Use of these terms is not intended to confer a specific order or orientation of the moiety unless explicitly so stated.

The terms “anti-CEACAM5 antibody” and “an antibody that binds to CEACAM5” refer to an antibody that is capable of binding CEACAM5 with sufficient affinity such that the antibody is useful as a diagnostic and/or therapeutic agent in targeting CEACAM5. In some aspects, the extent of binding of an anti-CEACAM5 antibody to an unrelated, non-CEACAM5 protein is less than about 10% of the binding of the antibody to CEACAM5 as measured, e.g., by surface plasmon resonance (SPR). In certain aspects, an antibody that binds to CEACAM5 has a dissociation constant (K_{D}) of ≤1 μM, ≤500 nM, ≤200 nM, ≤100 nM, ≤10 nM, ≤1 nM, ≤0.1 nM, ≤0.01 nM, or ≤0.001 nM, particularly a K_{D} of ≤1 nM, as measured by SPR at 25° C.

By “specific binding” is meant that the binding is selective for the antigen and can be discriminated from unwanted or non-specific interactions. Suitable assays for determining the specificity of the antibody of the present invention are described herein, including in the Examples hereinbelow. In some aspects, the extent of binding of an antibody to an unrelated protein, including a different member of the CEA family (particularly CEACAM1 or CEACAM6), is less than about 10% of the binding of the antibody to the antigen as measured, e.g., by SPR (using recombinant antigen) or FACS (using cells expressing the antigen).

The term “antibody” encompasses various antibody structures exhibiting the desired antigen-binding activity, including but not limited to monoclonal antibodies, polyclonal antibodies, multispecific antibodies (e.g. bispecific antibodies), and antibody fragments.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv and scFab), single-domain antibodies, and multispecific antibodies formed from antibody fragments. For a review of certain antibody fragments, see Hollinger and Hudson, Nature Biotechnology 23:1126-1136 (2005).

The terms “full-length antibody”, “intact antibody,” and “whole antibody” are used herein interchangeably to refer to an antibody having a structure substantially similar to a native antibody structure.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For review of methods for assessment of antibody purity, see, e.g., Flatman et al., J. Chromatogr. B 848:79-87 (2007). In some aspects, the antibodies provided by the present invention are isolated antibodies.

A “humanized” antibody refers to an antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody (the “parental” antibody), and all or substantially all of the FRs correspond to those of a human antibody. Such variable domains are referred to herein as “humanized variable region”. A humanized antibody optionally may comprise at least a portion of an antibody constant region derived from a human antibody. In some aspects, some FR residues in a humanized antibody are substituted with corresponding residues from a non-human antibody (e.g., the antibody from which the CDR residues are derived, i.e. the parental antibody), e.g., to restore or improve antibody specificity or affinity. The term “humanized” antibody also encompasses antibodies comprising certain mutations (e.g. amino acid substitutions) in the CDRs as compared to the CDRs of the parental antibody. In certain aspects, a humanized antibody comprises up to six (i.e. none, one, two, three, four, five or six) amino acid substitutions in one or more of its CDRs as compared to the parental antibody. In certain aspects, a humanized antibody comprises at least one CDR which is identical (i.e. does not comprise any amino acid substitutions as compared to the parental antibody) to the corresponding CDR of the parental antibody. In certain aspects, a humanized antibody comprises at least three CDRs (particularly at least one heavy chain CDR—more particularly at least HCDR3—and at least one light chain CDR) which are identical to the corresponding CDRs of the parental antibody. A “humanized form” of an antibody, e.g. of a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain aspects, a human antibody is derived from a non-human transgenic mammal, for example a mouse, a rat, or a rabbit. In certain aspects, a human antibody is derived from a hybridoma cell line. Antibodies or antibody fragments isolated from human antibody libraries are also considered human antibodies or human antibody fragments herein.

The term “antigen binding domain” refers to the part of an antibody that comprises the area which binds to and is complementary to part or all of an antigen. An antigen binding domain may be provided by, for example, one or more antibody variable domains (also called antibody variable regions). In preferred aspects, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6^{th} ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable regions or variable domains disclosed herein which comprise either a glutamine (Q) or a glutamate (E) amino acid residue at the N-terminus of an the antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N terminal pyro-glutamate (pyroE) residue instead of the N-terminal Q or E residue. Accordingly, for each antibody heavy chain, light chain, or variable domain or region sequence disclosed herein that contains an N-terminal Q or E residue, the corresponding sequence with an N-terminal pyroE residue is also encompassed.

Unless otherwise indicated, CDR residues and other residues in the variable domain (e.g., FR residues) are numbered herein according to Kabat et al., Sequences of Proteins of Immunological Interest, 5th Ed. Public Health Service, National Institutes of Health, Bethesda, MD (1991).

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (HCDR1, HCDR2, HCDR3), and three in the VL (LCDR1, LCDR2, LCDR3). CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis:

http://www.abysis.org/abysis/sequence_input/key_annotation/key_annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (H2), and 96-101 (H3), according to Chothia and Lesk, J. Mol. Biol. 196:901-917 (1987) (“Chothia definition”);
(b) CDRs occurring at amino acid residues 24-34 (L1), 50-56 (L2), 89-97 (L3), 31-35bB (H1), 50-65 (H2), and 95-102 (H3), according to Kabat et al., Sequences of Proteins of Immunological Interest, 5th Ed. Public Health Service, National Institutes of Health, Bethesda, MD (1991) (“Kabat definition”);
(c) antigen contacts occurring at amino acid residues 3027c-36 (L1), 46-55 (L2), 89-96 (L3), 30-35b (H1), 47-58 (H2), and 93-101 (H3), according to MacCallum et al. J. Mol. Biol. 262:732-745 (1996) (“Contact definition”); and
(d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27:55-77 (2003) (“IMGT definition”).

Unless otherwise indicated, the CDRs are determined herein according to Kabat et al., supra. One of skill in the art will understand that the CDR designations can also be determined according to Chothia, supra, MacCallum, supra, Lefranc, supra, or any other scientifically accepted definition/system.

“Framework” or “FR” refers to variable domain residues other than complementarity determining regions (CDRs). The FR of a variable domain generally consists of four FR domains: FR1, FR2, FR3, and FR4. Accordingly, the CDR and FR sequences generally appear in the following order in VH (or VL): FR1-HCDR1 (LCDR1)-FR2-HCDR2 (LCDR2)-FR3-HCDR3 (LCDR3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes is 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3.

The terms “constant region derived from human origin” or “human constant region” as used in the current application denotes a constant heavy chain region and/or a constant light chain (kappa or lambda) region of a human antibody, particularly a human antibody of the subclass IgG1, IgG2, IgG3, or IgG4. Such constant regions are well known in the state of the art and e.g. described by Kabat, E. A., et al., Sequences of Proteins of Immunological Interest, 5^{th} ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the numbering system as described in Kabat, E. A. et al., Sequences of Proteins of Immunological Interest, 5th ed., Public Health Service, National Institutes of Health, Bethesda, MD (1991), NIH Publication 91-3242. Specifically, the Kabat numbering system (referred to as “numbering according to Kabat” or “Kabat numbering” herein; see pages 647-660 of Kabat et al., supra) is used for the light chain constant domain of kappa and lambda isotype, and the Kabat EU index numbering system (referred to as “numbering according to Kabat EU index” or “Kabat EU index numbering” herein, see pages 661-723 of Kabat et al., supra) is used for the heavy chain constant domains.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types (or classes), called α (IgA), δ (IgD), ε (IgE), γ (IgG), or μ (IgM), some of which may be further divided into subtypes (or subclasses), e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Native antibodies” refers to naturally occurring immunoglobulin molecules with varying structures. For example, native IgG antibodies are immunoglobulin molecules of the IgG class.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

A “Fab molecule” or “Fab fragment” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “Fc domain” or “Fc region” (used interchangeably) herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. Amino acid sequences of heavy chains including an Fc region (or a subunit of an Fc domain as defined herein) are denoted herein without C-terminal glycine-lysine dipeptide if not indicated otherwise. In one aspect, a heavy chain including an Fc region (subunit) as specified herein, comprised in an antibody according to the invention, comprises an additional C-terminal glycine-lysine dipeptide (G446 and K447, numbering according to Kabat EU index). In one aspect, a heavy chain including an Fc region (subunit) as specified herein, comprised in an antibody according to the invention, comprises an additional C-terminal glycine residue (G446, numbering according to Kabat EU index). Unless otherwise specified herein, numbering of amino acid residues in the Fc region or heavy chain constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

The term “multispecific” means that the antibody is able to specifically bind to at least two distinct antigenic determinants. A multispecific antibody can be, for example, a bispecific antibody. Typically, a bispecific antibody comprises two antigen binding sites, each of which is specific for a different antigenic determinant. In certain aspects the multispecific (e.g. bispecific) antibody is capable of simultaneously binding two antigenic determinants, particularly two antigenic determinants expressed on two distinct cells.

The term “valent” as used herein denotes the presence of a specified number of antigen binding sites in an antibody. As such, e.g. the term “monovalent” denotes the presence of one (and not more than one) antigen binding site specific for the antigen in the antibody.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigenic determinant” or “antigen” refers to a site (e.g. a contiguous stretch of amino acids or a conformational configuration made up of different regions of non-contiguous amino acids) on a polypeptide macromolecule to which an antigen binding domain binds, forming an antigen binding domain-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). In a preferred aspect, the antigen is a human protein.

The term “T cell antigen” refers to an antigen expressed on the surface T lymphocyte. An exemplary T cell antigens is CD3.

“CD3” (Cluster of Differentiation 3) refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In a particular aspect, CD3 is human CD3, particularly the epsilon subunit of human CD3 (CD3E). The amino acid sequence of human CD38 is shown in SEQ ID NO: 96 (without signal peptide). See also UniProt (www.uniprot.org) accession no. P07766 (entry version 225), or NCBI (www.ncbi.nlm.nih.gov/) RefSeq NP_000724.1. In another aspect, CD3 is cynomolgus (Macaca fascicularis) CD3, particularly cynomolgus CD3E. The amino acid sequence of cynomolgus CD38 is shown in SEQ ID NO: 97 (without signal peptide). See also NCBI GenBank no. BAB71849.1.

“CEACAM5” stands for carcinoembryonic antigen-related cell adhesion molecule 5 (sometimes also just referred to as carcinoembryonic antigen or “CEA”) and refers to any native CEACAM5 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CEACAM5 as well as any form of CEACAM5 that results from processing in the cell. The term also encompasses naturally occurring variants of CEACAM5, e.g., splice variants or allelic variants. In particular aspects, CEACAM5 is membrane-bound CEACAM5. In further particular aspects, CEACAM5 is human CEACAM5. See for the human protein UniProt (www.uniprot.org) accession no. P06731 (version 195), or NCBI (www.ncbi.nlm.nih.gov/) RefSeq NP_004354.2. In preferred aspects, the antibody binds to human CEACAM5. In further preferred aspects, the antibody binds to membrane-bound CEACAM5.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (K_{D}). Affinity can be measured by well-established methods known in the art, including those described herein. A preferred method for measuring affinity is Surface Plasmon Resonance (SPR).

An “affinity matured” antibody refers to an antibody with one or more alterations in one or more complementarity determining regions (CDRs), compared to a parent antibody which does not possess such alterations, such alterations resulting in an improvement in the affinity of the antibody for antigen.

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero) dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In some aspects, the modification promoting the association of the first and the second subunit of the Fc domain comprises an amino acid mutation in the Fc domain, specifically an amino acid substitution. In a preferred aspect, the modification promoting the association of the first and the second subunit of the Fc domain comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B-cell receptor), and B-cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-258), and Pearson et. al. (Genomics 46 (1997) 24-36) and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

An “immunoconjugate” is an antibody conjugated to one or more heterologous molecule(s), including but not limited to a cytotoxic agent. Conversely, a “naked antibody” refers to an antibody that is not conjugated to a heterologous moiety (e.g., a cytotoxic moiety) or radiolabel. The naked antibody may be present in a pharmaceutical composition.

The term “polynucleotide” or “nucleic acid molecule” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2101823 B1).

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide (or nucleic acid) encoding an antibody” refers to one or more polynucleotide molecules encoding antibody heavy and light chains (or fragments thereof), including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antibodies of the present invention. Host cells include cultured cells, e.g. mammalian cultured cells, such as HEK cells, CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one aspect, the host cell of the invention is a eukaryotic cell, particularly a mammalian cell. In one aspect, the host cell is an isolated host cell. In one aspect, the host cell is not a cell within a human body.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human.

An “effective amount” of an agent, e.g., a pharmaceutical composition, refers to an amount effective, at dosages and for periods of time necessary, to achieve the desired therapeutic or prophylactic result.

The term “package insert” is used to refer to instructions customarily included in commercial packages of therapeutic products, that contain information about the indications, usage, dosage, administration, combination therapy, contraindications and/or warnings concerning the use of such therapeutic products.

### II. Compositions and Methods

The invention provides antibodies that bind to CEACAM5, including multispecific antibodies that bind to CEACAM5 and a second antigen. The antibodies of the invention show good produceability, combined with other favorable properties for therapeutic application, e.g. with respect to immunogenicity, affinity, specificity and efficacy. Antibodies of the invention are useful, e.g., for the treatment of diseases such as cancer.

### A. Anti-CEACAM5 Antibodies

In one aspect, the invention provides antibodies that bind to CEACAM5. In one aspect, provided are isolated antibodies that bind to CEACAM5. In one aspect, the invention provides antibodies that specifically bind to CEACAM5.

In one aspect, the invention provides an antibody that binds to CEACAM5, wherein the antibody comprises a heavy chain variable region (VH_{CEACAM5}) comprising the heavy chain complementarity determining region (HCDR) 1 of SEQ ID NO: 1, the HCDR 2 of SEQ ID NO: 12, and the HCDR 3 of SEQ ID NO: 3, and a light chain variable region (VL_{CEACAM5}) comprising the light chain complementarity determining region (LCDR) 1 of SEQ ID NO: 5, the LCDR 2 of SEQ ID NO: 26 and the LCDR 3 of SEQ ID NO: 28.

In some aspects, the VH_{CEACAM5} comprises the heavy chain complementarity determining region (HCDR) 1 of SEQ ID NO: 1, the HCDR 2 of SEQ ID NO: 12, and the HCDR 3 of SEQ ID NO: 3, and a light chain variable region (VL_{CEACAM5}) comprising the light chain complementarity determining region (LCDR) 1 of SEQ ID NO: 5, the LCDR 2 of SEQ ID NO: 27 and the LCDR 3 of SEQ ID NO: 28.

In some aspects,
(i) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 20;
(ii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 16;
(iii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 18;
(iv) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 22;
(v) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 7;
(vi) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 24, and the LCDR3 of SEQ ID NO: 7; or
(vii) the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 10, and the LCDR3 of SEQ ID NO: 7.

In particular aspects, the VH_{CEACAM5} comprises the HCDR1 of SEQ ID NO: 1, the HCDR2 of SEQ ID NO: 12, and the HCDR3 of SEQ ID NO: 3; and the VL_{CEACAM5} comprises the LCDR1 of SEQ ID NO: 5, the LCDR2 of SEQ ID NO: 14, and the LCDR3 of SEQ ID NO: 20.

In a further aspect, the invention provides an antibody that binds to CEACAM5, wherein the antibody comprises a heavy chain variable region (VH_{CEACAM5}) comprising the heavy chain complementarity determining region (HCDR) 1 of SEQ ID NO: 1, the HCDR 2 of SEQ ID NO: 12, and the HCDR 3 of SEQ ID NO: 3, and a light chain variable region (VL_{CEACAM5}) comprising the light chain complementarity determining region (LCDR) 1 of SEQ ID NO: 43, the LCDR 2 of SEQ ID NO: 10 and the LCDR 3 of SEQ ID NO: 7.

In some aspects, the antibody is a humanized antibody. In some aspects, the VH_{CEACAM5} and/or the VL_{CEACAM5} is a humanized variable region. In some aspects, the VH_{CEACAM5} and/or the VL_{CEACAM5} comprises an acceptor human framework, e.g. a human immunoglobulin framework or a human consensus framework. In certain aspects, the humanized antibody is derived from a parental antibody comprising the heavy chain variable region sequence of SEQ ID NO: 4 and the light chain variable region sequence of SEQ ID NO: 8.

In certain aspects, the antibody shows increased expression yield as compared to the parental antibody. In particular aspects, the antibody shows increased expression yield as compared to an antibody comprising the heavy chain variable region sequence of SEQ ID NO: 9 and the light chain variable region sequence of SEQ ID NO: 11. In some aspects, the antibody shows at least about 2-fold, at least about 3-fold, at least about 4-fold or at least about 5-fold, particularly at least about 3-fold, increased expression yield as compared to an antibody comprising the heavy chain variable region sequence of SEQ ID NO: 9 and the light chain variable region sequence of SEQ ID NO: 11. In specific aspects, such expression yield is in a mammalian expression system, particularly in CHO or HEK293 cells, most particularly in HEK293 cells. In further specific aspects, such expression yield is after Protein A affinity chromatography and/or size exclusion chromatography.

In some aspects, the antibody binds to CEACAM5 with essentially equal affinity as the parental antibody. In some aspects, the antibody binds to CEACAM5 with essentially equal affinity as an antibody comprising the heavy chain variable region sequence of SEQ ID NO: 9 and the light chain variable region sequence of SEQ ID NO: 11. In some aspects, the antibody binds to CEACAM5 with an affinity between about 0.5-fold and about 2-fold the affinity of an antibody comprising the heavy chain variable region sequence of SEQ ID NO: 9 and the light chain variable region sequence of SEQ ID NO: 11. In some aspects, the affinity is a K_{D} value. In some aspects, the affinity is to human CEACAM5 as measured by surface plasmon resonance (SPR) at 25° C. In other aspects, the affinity is to human CEACAM5 expressed on mammalian cells as measured by flow cytometry. In some aspects, the antibody binds to CEACAM5 with a K_{D} of 1 nM or less, as measured by surface plasmon resonance at 25° C.

In some aspects, the VH_{CEACAM5} comprises one or more heavy chain framework sequence (i.e. the FR1, FR2, FR3 and/or FR4 sequence) of the heavy chain variable region sequence of SEQ ID NO: 13.

In some aspects, the VH_{CEACAM5} comprises one or more heavy chain framework sequence selected from (a) the heavy chain framework region (H-FR) 1 of SEQ ID NO: 31, (b) the H-FR2 of SEQ ID NO: 32, (c) the H-FR3 of SEQ ID NO: 33, and (d) the H-FR4 of SEQ ID NO: 34.

In some aspects, the VH_{CEACAM5} comprises a H-FR1 of SEQ ID NO: 31. In some aspects, the VH_{CEACAM5} comprises a H-FR2 of SEQ ID NO: 32. In some aspects, the VH_{CEACAM5} comprises a H-FR3 of SEQ ID NO: 33. In some aspects, the VH_{CEACAM5} comprises a H-FR4 of SEQ ID NO: 34.

In some aspects, the VH_{CEACAM5} comprises a H-FR1 of at least 80%, 85%, 90% or 95% sequence identity to SEQ ID NO: 31. In some aspects, the VH_{CEACAM5} comprises a H-FR1 of at least 90% sequence identity to SEQ ID NO: 31. In some aspects, the VH_{CEACAM5} comprises a H-FR1 of at least 95% sequence identity to SEQ ID NO: 31.

In some aspects, the VH_{CEACAM5} comprises a H-FR2 of at least 80%, 85%, 90% or 95% sequence identity to SEQ ID NO: 32. In some aspects, the VH_{CEACAM5} comprises a H-FR2 of at least 80% sequence identity to SEQ ID NO: 32. In some aspects, the VH_{CEACAM5} comprises a H-FR2 of at least 90% sequence identity to SEQ ID NO: 32.

In some aspects, the VH_{CEACAM5} comprises a H-FR3 of at least 80%, 85%, 90% or 95% sequence identity to SEQ ID NO: 33. In some aspects, the VH_{CEACAM5} comprises a H-FR3 of at least 90% sequence identity to SEQ ID NO: 33. In some aspects, the VH_{CEACAM5} comprises a H-FR3 of at least 95% sequence identity to SEQ ID NO: 33.

In some aspects, the VH_{CEACAM5} comprises a H-FR4 of at least 80%, 85%, 90% or 95% sequence identity to SEQ ID NO: 34. In some aspects, the VH_{CEACAM5} comprises a H-FR4 of at least 80% sequence identity to SEQ ID NO: 34. In some aspects, the VH_{CEACAM5} comprises a H-FR4 of at least 90% sequence identity to SEQ ID NO: 34.

In some aspects, the VH_{CEACAM5} comprises a heavy chain framework region (H-FR) 1 comprising an amino acid sequence that is at least about 80%, 85%, 90%, 95% or 100% identical to the amino acid sequence of SEQ ID NO: 31, a H-FR2 comprising an amino acid sequence that is at least about 80%, 85%, 90%, 95% or 100% identical to the amino acid sequence of SEQ ID NO: 32, a H-FR3 comprising an amino acid sequence that is at least about 80%, 85%, 90%, 95% or 100% identical to the amino acid sequence of SEQ ID NO: 33, and/or a H-FR4 comprising an amino acid sequence that is at least about 80%, 85%, 90%, 95% or 100% identical to the amino acid sequence of SEQ ID NO: 34. In some aspects, the VH_{CEACAM5} comprises a H-FR1 comprising the amino acid sequence of SEQ ID NO: 31, a H-FR2 comprising the amino acid sequence of SEQ ID NO: 32, a H-FR3 comprising the amino acid sequence of SEQ ID NO: 33, and a H-FR4 comprising the amino acid sequence of SEQ ID NO: 34.

In some aspects, the VL_{CEACAM5} comprises one or more light chain framework sequence (i.e. the FR1, FR2, FR3 and/or FR4 sequence) of the light chain variable region sequence of SEQ ID NO: 29. In some aspects, the VL_{CEACAM5} comprises one or more light chain framework sequence (i.e. the FR1, FR2, FR3 and/or FR4 sequence) of the light chain variable region sequence of SEQ ID NO: 30. In some aspects, the VL_{CEACAM5} comprises one or more light chain framework sequence (i.e. the FR1, FR2, FR3 and/or FR4 sequence) of a light chain variable region sequence of SEQ ID NO: 21, 17, 19, 23, 15, 25 or 11. In some aspects, the VL_{CEACAM5} comprises one or more light chain framework sequence (i.e. the FR1, FR2, FR3 and/or FR4 sequence) of a light chain variable region sequence of SEQ ID NO: 44.

In some aspects, the VL_{CEACAM5} comprises one or more light chain framework sequence selected from (a) the light chain framework region (L-FR) 1 of SEQ ID NO: 35, (b) the L-FR2 of SEQ ID NO: 36, (c) the L-FR3 of SEQ ID NO: 37, and (d) the L-FR4 of SEQ ID NO: 38.

In some aspects, the VL_{CEACAM5} comprises a L-FR1 of SEQ ID NO: 35. In some aspects, the VL_{CEACAM5} comprises a L-FR2 of SEQ ID NO: 36. In some aspects, the VL_{CEACAM5} comprises a L-FR3 of SEQ ID NO: 37. In some aspects, the VL_{CEACAM5} comprises a L-FR4 of SEQ ID NO: 38.

In some aspects, the VL_{CEACAM5} comprises a L-FR1 of at least 80%, 85%, 90% or 95% sequence identity to SEQ ID NO: 35. In some aspects, the VL_{CEACAM5} comprises a L-FR1 of at least 90% sequence identity to SEQ ID NO: 35. In some aspects, the VL_{CEACAM5} comprises a L-FR1 of at least 95% sequence identity to SEQ ID NO: 35.

In some aspects, the VL_{CEACAM5} comprises a L-FR2 of at least 80%, 85%, 90% or 95% sequence identity to SEQ ID NO: 36. In some aspects, the VL_{CEACAM5} comprises a L-FR2 of at least 80% sequence identity to SEQ ID NO: 36. In some aspects, the VL_{CEACAM5} comprises a L-FR2 of at least 90% sequence identity to SEQ ID NO: 36.

In some aspects, the VL_{CEACAM5} comprises a L-FR3 of at least 80%, 85%, 90% or 95% sequence identity to SEQ ID NO: 37. In some aspects, the VL_{CEACAM5} comprises a L-FR3 of at least 90% sequence identity to SEQ ID NO: 37. In some aspects, the VL_{CEACAM5} comprises a L-FR3 of at least 95% sequence identity to SEQ ID NO: 37.

In some aspects, the VL_{CEACAM5} comprises a L-FR4 of at least 80%, 85%, 90% or 95% sequence identity to SEQ ID NO: 38. In some aspects, the VL_{CEACAM5} comprises a L-FR4 of at least 80% sequence identity to SEQ ID NO: 38. In some aspects, the VL_{CEACAM5} comprises a L-FR4 of at least 90% sequence identity to SEQ ID NO: 38.

In some aspects, the VL_{CEACAM5} comprises a light chain framework region (L-FR) 1 comprising an amino acid sequence that is at least about 80%, 85%, 90%, 95% or 100% identical to the amino acid sequence of SEQ ID NO: 35, a L-FR2 comprising an amino acid sequence that is at least about 80%, 85%, 90%, 95% or 100% identical to the amino acid sequence of SEQ ID NO: 36, a L-FR3 comprising an amino acid sequence that is at least about 80%, 85%, 90%, 95% or 100% identical to the amino acid sequence of SEQ ID NO: 37, and/or a L-FR4 comprising an amino acid sequence that is at least about 80%, 85%, 90%, 95% or 100% identical to the amino acid sequence of SEQ ID NO: 38. In some aspects, the VL_{CEACAM5} comprises a L-FR1 comprising the amino acid sequence of SEQ ID NO: 35, a L-FR2 comprising the amino acid sequence of SEQ ID NO: 36, a L-FR3 comprising the amino acid sequence of SEQ ID NO: 37, and a L-FR4 comprising the amino acid sequence of SEQ ID NO: 38.

In some aspects, the VH_{CEACAM5} comprises an amino acid sequence that is at least about 95%, 96%, 97%, 98%, or 99% identical to the amino acid sequence of SEQ ID NO: 13. In some aspects, the VH_{CEACAM5} comprises an amino acid sequence that is at least about 95% identical to the amino acid sequence of SEQ ID NO: 13. In some aspects, the VH_{CEACAM5} comprises an amino acid sequence that is at least about 98% identical to the amino acid sequence of SEQ ID NO: 13.

In certain aspects, a VH sequence having at least 95%, 96%, 97%, 98%, or 99% identity contains substitutions (e.g., conservative substitutions), insertions, or deletions relative to the reference sequence, but an antibody comprising that sequence retains the ability to bind to CEACAM5. In certain aspects, a total of 1 to 10 amino acids have been substituted, inserted and/or deleted in the amino acid sequence of SEQ ID NO: 13. In certain aspects, substitutions, insertions, or deletions occur in regions outside the CDRs (i.e., in the FRs).

In some aspects, the VH_{CEACAM5} comprises the amino acid sequence of SEQ ID NO: 13. Optionally, the VH_{CEACAM5} comprises the amino acid sequence of SEQ ID NO: 13, including post-translational modifications of that sequence.

In some aspects, the VL_{CEACAM5} comprises an amino acid sequence that is at least about 95%, 96%, 97%, 98%, or 99% identical to the amino acid sequence of SEQ ID NO: 29. In some aspects, the VL_{CEACAM5} comprises an amino acid sequence that is at least about 95% identical to the amino acid sequence of SEQ ID NO: 29. In some aspects, the VL_{CEACAM5} comprises an amino acid sequence that is at least about 98% identical to the amino acid sequence of SEQ ID NO: 29. In some aspects, the VL_{CEACAM5} comprises an amino acid sequence that is at least about 95%, 96%, 97%, 98%, or 99% identical to the amino acid sequence of SEQ ID NO: 30. In some aspects, the VL_{CEACAM5} comprises an amino acid sequence that is at least about 95% identical to the amino acid sequence of SEQ ID NO: 30. In some aspects, the VL_{CEACAM5} comprises an amino acid sequence that is at least about 98% identical to the amino acid sequence of SEQ ID NO: 30.

In some aspects, the VL_{CEACAM5} comprises an amino acid sequence that is at least about 95%, 96%, 97%, 98%, or 99% identical to an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. In some aspects, the VL_{CEACAM5} comprises an amino acid sequence that is at least about 95% identical to an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. In some aspects, the VL_{CEACAM5} comprises an amino acid sequence that is at least about 98% identical to an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. In certain aspects, a VL sequence having at least 95%, 96%, 97%, 98%, or 99% identity contains substitutions (e.g., conservative substitutions), insertions, or deletions relative to the reference sequence, but an antibody comprising that sequence retains the ability to bind to CEACAM5.

In certain aspects, a total of 1 to 10 amino acids have been substituted, inserted and/or deleted in the amino acid sequence of SEQ ID NO: 29, SEQ ID NO: 30, SEQ ID NO: 21, SEQ ID NO: 17, SEQ ID NO: 19, SEQ ID NO: 23, SEQ ID NO: 15, SEQ ID NO: 25 or SEQ ID NO: 11. In certain aspects, substitutions, insertions, or deletions occur in regions outside the CDRs (i.e., in the FRs).

In some aspects, the VL_{CEACAM5} comprises the amino acid sequence of SEQ ID NO: 29. Optionally, the VL_{CEACAM5} comprises the amino acid sequence of SEQ ID NO: 29, including post-translational modifications of that sequence. In some aspects, the VL_{CEACAM5} comprises the amino acid sequence of SEQ ID NO: 30. Optionally, the VL_{CEACAM5} comprises the amino acid sequence of SEQ ID NO: 30, including post-translational modifications of that sequence.

In some aspects, the VL_{CEACAM5} comprises an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. Optionally, the VL_{CEACAM5} comprises an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21, including post-translational modifications of that sequence.

In other aspects, the VL_{CEACAM5} comprises an amino acid sequence that is at least about 95%, 96%, 97%, 98%, or 99% identical to the amino acid sequence of SEQ ID NO: 44. In some aspects, the VL_{CEACAM5} comprises an amino acid sequence that is at least about 95% identical to the amino acid sequence of SEQ ID NO: 44. In some aspects, the VL_{CEACAM5} comprises an amino acid sequence that is at least about 98% identical to the amino acid sequence of SEQ ID NO: 44. In certain aspects, a VL sequence having at least 95%, 96%, 97%, 98%, or 99% identity contains substitutions (e.g., conservative substitutions), insertions, or deletions relative to the reference sequence, but an antibody comprising that sequence retains the ability to bind to CEACAM5. In certain aspects, a total of 1 to 10 amino acids have been substituted, inserted and/or deleted in the amino acid sequence of SEQ ID NO: 44. In certain aspects, substitutions, insertions, or deletions occur in regions outside the CDRs (i.e., in the FRs).

In some aspects, the VL_{CEACAM5} comprises the amino acid sequence of SEQ ID NO: 44. Optionally, the VL_{CEACAM5} comprises the amino acid sequence of SEQ ID NO: 44, including post-translational modifications of that sequence.

In one aspect, the anti-CEACAM5 antibody comprises a VH sequence as in any of the aspects provided above, and a VL sequence as in any of the aspects provided above.

In some aspects, the VH_{CEACAM5} comprises an amino acid sequence that is at least about 95%, 96%, 97%, 98% or 99% identical to the amino acid sequence of SEQ ID NO: 13; and/or the VL_{CEACAM5} comprises an amino acid sequence that is at least about 95%, 96%, 97%, 98% or 99% identical to the amino acid sequence of SEQ ID NO: 29. In some aspects, the VH_{CEACAM5} comprises the amino acid sequence of SEQ ID NO: 13; and/or the VL_{CEACAM5} comprises the amino acid sequence of SEQ ID NO: 29. In some aspects, the VH_{CEACAM5} comprises an amino acid sequence that is at least about 95%, 96%, 97%, 98% or 99% identical to the amino acid sequence of SEQ ID NO: 13; and/or the VL_{CEACAM5} comprises an amino acid sequence that is at least about 95%, 96%, 97%, 98% or 99% identical to the amino acid sequence of SEQ ID NO: 30. In some aspects, the VH_{CEACAM5} comprises the amino acid sequence of SEQ ID NO: 13; and/or the VL_{CEACAM5} comprises the amino acid sequence of SEQ ID NO: 30.

In some aspects, the VH_{CEACAM5} comprises an amino acid sequence that is at least about 95%, 96%, 97%, 98% or 99% identical to the amino acid sequence of SEQ ID NO: 13; and/or the VL_{CEACAM5} comprises an amino acid sequence that is at least about 95%, 96%, 97%, 98% or 99% identical to an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. In some aspects, the VH_{CEACAM5} comprises the amino acid sequence of SEQ ID NO: 13; and/or the VL_{CEACAM5} comprises an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21.

In some aspects,
(i) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 21;
(ii) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 17;
(iii) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 19;
(iv) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 23;
(v) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 15;
(vi) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 25; or
(vii) the VH_{CEACAM5} comprises an amino acid sequence that is at least about 95%, 96%, 97%, 98% or 99% identical to the amino acid sequence of SEQ ID NO: 13 and/or the VL_{CEACAM5} comprises an amino acid sequence that is at least about 95%, 96%, 97%, 98% or 99% identical to the amino acid sequence of SEQ ID NO: 11.

In some aspects,
(i) the VH_{CEACAM5} comprises the amino acid sequence of SEQ ID NO: 13 and/or the VL_{CEACAM5} comprises the amino acid sequence of SEQ ID NO: 21;
(ii) the VH_{CEACAM5} comprises the amino acid sequence of SEQ ID NO: 13 and/or the VL_{CEACAM5} comprises the amino acid sequence of SEQ ID NO: 17;
(iii) the VH_{CEACAM5} comprises the amino acid sequence of SEQ ID NO: 13 and/or the VL_{CEACAM5} comprises the amino acid sequence of SEQ ID NO: 19;
(iv) the VH_{CEACAM5} comprises the amino acid sequence of SEQ ID NO: 13 and/or the VL_{CEACAM5} comprises the amino acid sequence of SEQ ID NO: 23;
(v) the VH_{CEACAM5} comprises the amino acid sequence of SEQ ID NO: 13 and/or the VL_{CEACAM5} comprises the amino acid sequence of SEQ ID NO: 15;
(vi) the VH_{CEACAM5} comprises the amino acid sequence of SEQ ID NO: 13 and/or the VL_{CEACAM5} comprises the amino acid sequence of SEQ ID NO: 25;
(vii) the VH_{CEACAM5} comprises the amino acid sequence of SEQ ID NO: 13 and/or the VL_{CEACAM5} comprises the amino acid sequence of SEQ ID NO: 11.

In particular aspects, the VH_{CEACAM5} comprises an amino acid sequence that is at least about 95%, 96%, 97%, 98% or 99% identical to the amino acid sequence of SEQ ID NO: 13 and the VL_{CEACAM5} comprises an amino acid sequence that is at least about 95%, 96%, 97%, 98% or 99% identical to the amino acid sequence of SEQ ID NO: 21. In further particular aspects, the VH_{CEACAM5} comprises the amino acid sequence of SEQ ID NO: 13 and the VL_{CEACAM5} comprises the amino acid sequence of SEQ ID NO: 21.

In other aspects, the VH_{CEACAM5} comprises an amino acid sequence that is at least about 95%, 96%, 97%, 98% or 99% identical to the amino acid sequence of SEQ ID NO: 13 and the VL_{CEACAM5} comprises an amino acid sequence that is at least about 95%, 96%, 97%, 98% or 99% identical to the amino acid sequence of SEQ ID NO: 44. In further particular aspects, the VH_{CEACAM5} comprises the amino acid sequence of SEQ ID NO: 13 and the VL_{CEACAM5} comprises the amino acid sequence of SEQ ID NO: 44.

In another aspect, the invention provides an antibody that binds to CEACAM5, wherein the antibody comprises a VH_{CEACAM5} sequence as in any of the aspects provided above, and a VH_{CEACAM5} sequence as in any of the aspects provided above. In some aspects, the antibody comprises the VH_{CEACAM5} and VL_{CEACAM5} sequences in SEQ ID NO: 13 and SEQ ID NO: 29, respectively, including post-translational modifications of those sequences. In some aspects, the antibody comprises the VH_{CEACAM5} and VL_{CEACAM5} Sequences in SEQ ID NO: 13 and SEQ ID NO: 30, respectively, including post-translational modifications of those sequences. In some aspects, the antibody comprises the VH_{CEACAM5} and VL_{CEACAM5} sequences in SEQ ID NO: 13 and SEQ ID NO: 21, 17, 19, 23, 15, 25 or 11 (particularly SEQ ID NO: 21), respectively, including post-translational modifications of those sequences.

In other aspects, the antibody comprises the VH_{CEACAM5} and VL_{CEACAM5} sequences in SEQ ID NO: 13 and SEQ ID NO: 44, respectively, including post-translational modifications of those sequences.

In one aspect, the invention provides an antibody that binds to CEACAM5, comprising a heavy chain variable region (VH_{CEACAM5}) comprising an amino acid sequence that is at least about 95%, 96%, 97%, 98% or 99% identical to the amino acid sequence of SEQ ID NO: 13, and/or a light chain variable region (VL_{CEACAM5}) comprising an amino acid sequence that is at least about 95%, 96%, 97%, 98% or 99% identical to the amino acid sequence of SEQ ID NO: 29. In some aspects, the VH_{CEACAM5} comprises the amino acid sequence of SEQ ID NO: 13 and/or the VL_{CEACAM5} comprises the amino acid sequence of SEQ ID NO: 29. In one aspect, the invention provides an antibody that binds to CEACAM5, comprising a heavy chain variable region (VH_{CEACAM5}) comprising an amino acid sequence that is at least about 95%, 96%, 97%, 98% or 99% identical to the amino acid sequence of SEQ ID NO: 13, and/or a light chain variable region (VL_{CEACAM5}) comprising an amino acid sequence that is at least about 95%, 96%, 97%, 98% or 99% identical to the amino acid sequence of SEQ ID NO: 30. In some aspects, the VH_{CEACAM5} comprises the amino acid sequence of SEQ ID NO: 13 and/or the VL_{CEACAM5} comprises the amino acid sequence of SEQ ID NO: 30.

In another aspect, the invention provides an antibody that binds to CEACAM5, comprising a heavy chain variable region (VH_{CEACAM5}) comprising an amino acid sequence that is at least about 95%, 96%, 97%, 98% or 99% identical to the amino acid sequence of SEQ ID NO: 13, and/or a light chain variable region (VL_{CEACAM5}) comprising an amino acid sequence that is at least about 95%, 96%, 97%, 98% or 99% identical to an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21. In some aspects, the VH_{CEACAM5} comprises the amino acid sequence of SEQ ID NO: 13, and/or the VL_{CEACAM5} comprises an amino acid sequence selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the amino acid sequence of SEQ ID NO: 21.

In one aspect, the invention provides an antibody that binds to CEACAM5, comprising
(i) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 21;
(ii) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 17;
(iii) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 19;
(iv) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 23;
(v) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 15;
(vi) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 25; or
(vii) a heavy chain variable region (VH_{CEACAM5}) comprising an amino acid sequence that is at least about 95%, 96%, 97%, 98% or 99% identical to the amino acid sequence of SEQ ID NO: 13 and/or a light chain variable region (VL_{CEACAM5}) comprising an amino acid sequence that is at least about 95%, 96%, 97%, 98% or 99% identical to the amino acid sequence of SEQ ID NO: 11.

In some aspects,
(i) the VH_{CEACAM5} comprises the amino acid sequence of SEQ ID NO: 13 and/or the VL_{CEACAM5} comprises the amino acid sequence of SEQ ID NO: 21;
(ii) the VH_{CEACAM5} comprises the amino acid sequence of SEQ ID NO: 13 and/or the VL_{CEACAM5} comprises the amino acid sequence of SEQ ID NO: 17;
(iii) the VH_{CEACAM5} comprises the amino acid sequence of SEQ ID NO: 13 and/or the VL_{CEACAM5} comprises the amino acid sequence of SEQ ID NO: 19;
(iv) the VH_{CEACAM5} comprises the amino acid sequence of SEQ ID NO: 13 and/or the VL_{CEACAM5} comprises the amino acid sequence of SEQ ID NO: 23;
(v) the VH_{CEACAM5} comprises the amino acid sequence of SEQ ID NO: 13 and/or the VL_{CEACAM5} comprises the amino acid sequence of SEQ ID NO: 15;
(vi) the VH_{CEACAM5} comprises the amino acid sequence of SEQ ID NO: 13 and/or the VL_{CEACAM5} comprises the amino acid sequence of SEQ ID NO: 25; or
(vii) the VH_{CEACAM5} comprises the amino acid sequence of SEQ ID NO: 13 and/or the VL_{CEACAM5} comprises the amino acid sequence of SEQ ID NO: 11.

In a particular aspect, the invention provides an antibody that binds to CEACAM5, comprising a heavy chain variable region (VH_{CEACAM5}) comprising an amino acid sequence that is at least about 95%, 96%, 97%, 98% or 99% identical to the amino acid sequence of SEQ ID NO: 13 and a light chain variable region (VL_{CEACAM5}) comprising an amino acid sequence that is at least about 95%, 96%, 97%, 98% or 99% identical to the amino acid sequence of SEQ ID NO: 21. In particular aspects, the VH_{CEACAM5} comprises the amino acid sequence of SEQ ID NO: 13 and the VL_{CEACAM5} comprises the amino acid sequence of SEQ ID NO: 21.

In another aspect, the invention provides an antibody that binds to CEACAM5, comprising a heavy chain variable region (VH_{CEACAM5}) comprising an amino acid sequence that is at least about 95%, 96%, 97%, 98% or 99% identical to the amino acid sequence of SEQ ID NO: 13 and a light chain variable region (VL_{CEACAM5}) comprising an amino acid sequence that is at least about 95%, 96%, 97%, 98% or 99% identical to the amino acid sequence of SEQ ID NO: 44. In particular aspects, the VH_{CEACAM5} comprises the amino acid sequence of SEQ ID NO: 13 and the VL_{CEACAM5} comprises the amino acid sequence of SEQ ID NO: 44.

In one aspect, the invention provides an antibody that binds to CEACAM5, comprising a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 29. In another aspect, the invention provides an antibody that binds to CEACAM5, comprising a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 30.

In one aspect, the invention provides an antibody that binds to CEACAM5, comprising a heavy chain variable region (VH_{CEACAM5}) comprising the heavy chain CDR sequences of the VH of SEQ ID NO: 13, and a light chain variable region (VL_{CEACAM5}) comprising the light chain CDR sequences of a VL selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the VL of SEQ ID NO: 21.

In a further aspect, the invention provides an antibody that binds to CEACAM5, comprising
(i) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 21;
(ii) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 17;
(iii) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 19;
(iv) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 23;
(v) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 15;
(vi) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 25; or
(vi) a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 11.

In a particular aspect, the invention provides an antibody that binds to CEACAM5, comprising a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 21.

In another aspect, the invention provides an antibody that binds to CEACAM5, comprising a heavy chain variable region (VH_{CEACAM5}) comprising the heavy chain CDR sequences of the VH of SEQ ID NO: 13, and a light chain variable region (VL_{CEACAM5}) comprising the light chain CDR sequences of the VL of SEQ ID NO: 44.

In some aspects, the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 29. In some aspects, the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 30.

In some aspects, the VH_{CEACAM5} comprises the HCDR1, HCDR2 and HCDR3 amino acid sequences of the VH selected of SEQ ID NO: 13, and the VL_{CEACAM5} comprises the LCDR1, LCDR2 and LCDR3 amino acid sequences of a VL selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the VL of SEQ ID NO: 21.

In some aspects,
(i) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 21;
(ii) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 17;
(iii) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 19;
(iv) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 23;
(v) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 15;
(vi) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 25; or
(vii) the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 11.

In particular aspects, the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 21.

In other aspects, the VH_{CEACAM5} comprises the HCDR1, HCDR2 and HCDR3 amino acid sequences of the VH of SEQ ID NO: 13, and the VL_{CEACAM5} comprises the LCDR1, LCDR2 and LCDR3 amino acid sequences of the VL of SEQ ID NO: 44.

In some aspects, the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 29, and a framework of at least 95%, 96%, 97%, 98% or 99%, particularly at least 95% or at least 98%, sequence identity to the framework sequence of the VL of SEQ ID NO: 29. In some aspects, the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 30, and a framework of at least 95%, 96%, 97%, 98% or 99%, particularly at least 95% or at least 98%, sequence identity to the framework sequence of the VL of SEQ ID NO: 30.

In some aspects, the VH_{CEACAM5} comprises the heavy chain CDR sequences of the VH selected of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of a VL selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the VL of SEQ ID NO: 21, and a framework of at least 95%, 96%, 97%, 98% or 99%, particularly at least 95% or at least 98%, sequence identity to the framework sequence of a VL selected from the group consisting of SEQ ID NO: 21, SEQ ID NO: 17, SEQ ID NO: 19, SEQ ID NO: 23, SEQ ID NO: 15, SEQ ID NO: 25 and SEQ ID NO: 11, particularly the VL of SEQ ID NO: 21.

In further aspects,
(i) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 21, and a framework of at least 95%, 96%, 97%, 98% or 99%, particularly at least 95% or at least 98%, sequence identity to the framework sequence of the VL of SEQ ID NO: 21;
(ii) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 17, and a framework of at least 95%, 96%, 97%, 98% or 99%, particularly at least 95% or at least 98%, sequence identity to the framework sequence of the VL of SEQ ID NO: 17;
(iii) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 19, and a framework of at least 95%, 96%, 97%, 98% or 99%, particularly at least 95% or at least 98%, sequence identity to the framework sequence of the VL of SEQ ID NO: 19;
(iv) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 23, and a framework of at least 95%, 96%, 97%, 98% or 99%, particularly at least 95% or at least 98%, sequence identity to the framework sequence of the VL of SEQ ID NO: 23;
(v) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 15, and a framework of at least 95%, 96%, 97%, 98% or 99%, particularly at least 95% or at least 98%, sequence identity to the framework sequence of the VL of SEQ ID NO: 15;
(vi) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 25, and a framework of at least 95%, 96%, 97%, 98% or 99%, particularly at least 95% or at least 98%, sequence identity to the framework sequence of the VL of SEQ ID NO: 25; or
(vii) the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 11, and a framework of at least 95%, 96%, 97%, 98% or 99%, particularly at least 95% or at least 98%, sequence identity to the framework sequence of the VL of SEQ ID NO: 11.

In particular aspects, the VH_{CEACAM5} comprises the heavy chain CDR sequences of the VH of SEQ ID NO: 13, and a framework of at least 95%, 96%, 97%, 98% or 99% sequence identity to the framework sequence of the VH of SEQ ID NO: 13. In some aspects, the VH_{CEACAM5} comprises the heavy chain CDR sequences of the VH of SEQ ID NO: 13, and a framework of at least 95% sequence identity to the framework sequence of the VH of SEQ ID NO: 13. In other aspects, the VH_{CEACAM5} comprises the heavy chain CDR sequences of the VH of SEQ ID NO: 13, and a framework of at least 98% sequence identity to the framework sequence of the VH of SEQ ID NO: 13.

In further particular aspects, the VL_{CEACAM5} comprises the light chain CDR sequences of the VL of SEQ ID NO: 21 and a framework of at least 95%, 96%, 97%, 98% or 99% sequence identity to the framework sequence of the VL of SEQ ID NO: 21. In some aspects, the VL_{CEACAM5} comprises the light chain CDR sequences of the VL of SEQ ID NO: 21 and a framework of at least 95% sequence identity to the framework sequence of the VL of SEQ ID NO: 21. In other aspects, the VL_{CEACAM5} comprises the light chain CDR sequences of the VL of SEQ ID NO: 21 and a framework of at least 98% sequence identity to the framework sequence of the VL of SEQ ID NO: 21.

In other aspects, the VH_{CEACAM5} comprises the heavy chain CDR sequences of the VH of SEQ ID NO: 13, and a framework of at least 95%, 96%, 97%, 98% or 99%, particularly at least 95% or at least 98%, sequence identity to the framework sequence of the VH of SEQ ID NO: 13, and/or the VL_{CEACAM5} comprises the light chain CDR sequences of the VL of SEQ ID NO: 44, and a framework of at least 95%, 96%, 97%, 98% or 99%, particularly at least 95% or at least 98%, sequence identity to the framework sequence of the VL of SEQ ID NO: 44.

Preferably, the CDR sequences of the VH and/or VL according to the above aspects are according to the Kabat definition. Alternatively, the CDRs of the VH and/or VL domain according to the above aspects are according to the Chothia definition. Further alternatively, the CDRs of the VH and/or VL domain according to the above aspects are according to the Contact definition. Still further alternatively, the CDRs of the VH and/or VL domain according to the above aspects are according to the IMGT definition.

In some aspects, the antibody comprises a human constant region. In some aspects, the antibody is an immunoglobulin molecule comprising a human constant region, particularly an IgG class, most particularly an IgG₁ class, immunoglobulin molecule comprising a human CH1, CH2, CH3 and/or CL domain. Exemplary sequences of human constant domains are given in SEQ ID NOs 40 and 41 (human kappa and lambda CL domains, respectively) and SEQ ID NO: 42 (human IgG1 heavy chain constant domains CH1-CH2-CH3). In some aspects, the antibody comprises a light chain constant region comprising an amino acid sequence that is at least about 95%, 96%, 97%, 98%, 99% or 100% identical to the amino acid sequence of SEQ ID NO: 40 or SEQ ID NO: 41, particularly the amino acid sequence of SEQ ID NO: 40. In some aspects, the antibody comprises a heavy chain constant region comprising an amino acid sequence that is at least about 95%, 96%, 97%, 98%, 99% or 100% identical to the amino acid sequence of SEQ ID NO: 42. Particularly, the heavy chain constant region may comprise amino acid mutations in the Fc region as described herein.

In some aspects, the antibody comprises an Fc region. The Fc region of the antibody consists of a pair of polypeptide chains comprising heavy chain domains of an immunoglobulin molecule. For example, the Fc region of an immunoglobulin G (IgG) molecule is a dimer, each subunit of which comprises the CH2 and CH3 IgG heavy chain constant domains. The two subunits of the Fc region are capable of stable association with each other. In particular aspects, the Fc region of the antibody provided herein is an IgG Fc region, more particularly an IgG₁ Fc region. In other aspects, the Fc region is an IgG₄ Fc region. In more specific aspects, the Fc region is an IgG₄ Fc region comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In particular aspects, the Fc region is a human Fc region. In even more particular aspects, the Fc region is a human IgG₁ Fc region. An exemplary sequence of a human IgG₁ Fc region is given in SEQ ID NO: 39.

In some aspects, the antibody is a monoclonal antibody.

In some aspects, the antibody is an IgG, particularly an IgG₁, antibody. In some aspects, the antibody is a full-length antibody. In some aspects, the antibody is an immunoglobulin molecule, particularly an immunoglobulin molecule of the IgG class, most particularly an immunoglobulin molecule of the IgG₁ subclass. In some aspects, the antibody comprises an Fc region, particularly an IgG Fc region, more particularly an IgG₁ Fc region. In some aspects the Fc region is a human Fc region. In some aspects, the Fc region is a human IgG₁ Fc region. In other aspects, the antibody is an antibody fragment that binds to CEACAM5, particularly an antibody fragment selected from the group of an Fv molecule, a scFv molecule, a Fab molecule, and a F(ab′)₂ molecule. In some aspects, the antibody fragment is a diabody, a triabody or a tetrabody.

In particular aspects, the antibody is a multispecific antibody, particularly a bispecific antibody.

In further aspects, the antibody according to any of the above aspects may incorporate any of the features, singly or in combination, as described in sections II. A. 1. -5. below.

### 1. Antibody Fragments

In certain aspects, an antibody provided herein is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” (or “Fab molecule”) thus refers to an antibody fragment comprising a light chain comprising a VL domain and a CL domain, and a heavy chain fragment comprising a VH domain and a CH1 domain. “Fab′ fragments” (or “Fab′ molecules”) differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” (or “scFab molecule”) is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” (or “scFv molecule”) is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

Antibody fragments can be made by various techniques, including but not limited to proteolytic digestion of an intact antibody as well as recombinant production by recombinant host cells (e.g., E. coli), as described herein.

### 2. Humanized Antibodies

In certain aspects, an antibody provided herein is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 3. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for CEACAM5 and the other specificity is for any other antigen. In certain aspects, bispecific antibodies may bind to two (or more) different epitopes of CEACAM5. Multispecific (e.g., bispecific) antibodies may also be used to localize cytotoxic agents or cells to cells which express CEACAM5. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to CEACAM5 as well as another different antigen, or two different epitopes of CEACAM5 (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein et al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for CEACAM5 and the other is for CD3 (i.e. the antibody is a bispecific antibody that binds to CEACAM5 and to CD3).

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) e1203498.

Particular aspects of the multispecific, specifically bispecific, antibodies of the present invention are described in the following.

In some aspects, the antibody comprises a first antigen binding domain which binds to CEACAM5 and comprises the VH_{CEACAM5} and the VL_{CEACAM5} (as defined in any of the above aspects), and a second antigen binding domain which binds to a second antigen. In some aspects, the second antigen is a T cell antigen. In some aspects, the second antigen is CD3, particularly CD3E. In some such aspects, the second antigen binding domain comprises a heavy chain variable region (VH_{CD3}) and a light chain variable region (VL_{CD3}). In some aspects, the VH_{CD3} comprises the HCDR1 of SEQ ID NO: 45, the HCDR2 of SEQ ID NO: 46, and the HCDR3 of SEQ ID NO: 47, and the VL_{CD3} comprises the LCDR1 of SEQ ID NO: 49, the LCDR2 of SEQ ID NO: 50, and the LCDR3 of SEQ ID NO: 51. In some aspects, the VH_{CD3} comprises an amino acid sequence that is at least about 95%, 96%, 97%, 98%, 99% or 100% identical to the amino acid sequence of SEQ ID NO: 48, and/or the VL_{CD3} comprises an amino acid sequence that is at least about 95%, 96%, 97%, 98%, 99% or 100% identical to the amino acid sequence of SEQ ID NO: 52.

In some aspects, the first and/or the second antigen binding domain is a Fab molecule. In some aspects, the second antigen binding domain is a Fab molecule wherein the variable domains VL and VH or the constant domains CL and CH1, particularly the variable domains VL and VH, of the Fab light chain and the Fab heavy chain are replaced by each other. In some aspects, the first antigen binding domain is a conventional Fab molecule. In some aspects, the first antigen binding domain is a Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In some aspects, the antibody comprises a third antigen binding domain. In some aspects, the third antigen binding domain binds to CEACAM5. In some aspects, the third antigen binding domain comprises a VH_{CEACAM5} and/or a VL_{CEACAM5} as defined in any of the above aspects. In some aspects, the third antigen binding domain is a Fab molecule. In some aspects, the third antigen binding domain is a conventional Fab molecule. In some aspects, the third antigen binding domain is a Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index). In some aspects, the third antigen binding domain is identical to the first antigen binding domain.

In some aspects, the first and the second antigen binding domain are fused to each other, optionally via a peptide linker. In some aspects, the first and the second antigen binding domain are each a Fab molecule and either (i) the first antigen binding domain is fused at the C-terminus of the Fab heavy chain to the N-terminus of the Fab heavy chain of the second antigen binding domain, or (ii) the second antigen binding domain is fused at the C-terminus of the Fab heavy chain to the N-terminus of the Fab heavy chain of the first antigen binding domain.

In some aspects, the antibody comprises an Fc domain composed of a first and a second subunit.

In some aspects, the first and the second antigen binding domain are each a Fab molecule and the antibody comprises an Fc domain composed of a first and a second subunit, and wherein the first antigen binding domain is fused at the C-terminus of the Fab heavy chain to the N-terminus of one of the subunits of the Fc domain, particularly the second subunit of the Fc domain, and the second antigen binding domain is fused at the C-terminus of the Fab heavy chain to the N-terminus of the other subunit of the Fc domain, particularly the first subunit of the Fc domain.

In some aspects, the first and the second antigen binding domain are each a Fab molecule and the antibody comprises an Fc domain composed of a first and a second subunit; and wherein either (i) the first antigen binding domain is fused at the C-terminus of the Fab heavy chain to the N-terminus of the Fab heavy chain of the second antigen binding domain and the second antigen binding domain is fused at the C-terminus of the Fab heavy chain to the N-terminus of one of the subunits of the Fc domain, particularly the first subunit of the Fc domain, or (ii) the second antigen binding domain is fused at the C-terminus of the Fab heavy chain to the N-terminus of the Fab heavy chain of the first antigen binding domain and the first antigen binding domain is fused at the C-terminus of the Fab heavy chain to the N-terminus of one of the subunits of the Fc domain, particularly the first subunit of the Fc domain.

In some aspects, the first, the second and the third antigen binding domain are each a Fab molecule and the antibody comprises an Fc domain composed of a first and a second subunit; and either (i) the first antigen binding domain is fused at the C-terminus of the Fab heavy chain to the N-terminus of the Fab heavy chain of the second antigen binding domain and the second antigen binding domain is fused at the C-terminus of the Fab heavy chain to the N-terminus of one of the subunits of the Fc domain, particularly the first subunit of the Fc domain, or (ii) the second antigen binding domain is fused at the C-terminus of the Fab heavy chain to the N-terminus of the Fab heavy chain of the first antigen binding domain and the first antigen binding domain is fused at the C-terminus of the Fab heavy chain to the N-terminus of one of the subunits of the Fc domain, particularly the first subunit of the Fc domain; and the third antigen binding domain is fused at the C-terminus of the Fab heavy chain to the N-terminus of the other subunit of the Fc domain, particularly the second subunit of the Fc domain.

In some aspects, the Fc domain is an IgG, particularly an IgG₁, Fc domain. In some aspects, the Fc domain is a human Fc domain. In particular aspects, the Fc domain is a human IgG₁ Fc domain.

In some aspects, the Fc comprises a modification promoting the association of the first and the second subunit of the Fc domain. In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in preferred aspects, in the CH3 domain of the first subunit of the Fc domain of the (multispecific) antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine(S), threonine (T), and valine (V).

The protuberance and cavity can be made by altering the nucleic acid encoding the polypeptides, e.g. by site-specific mutagenesis, or by peptide synthesis.

In specific aspects, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (γ₄₀₇V). In some aspects, in the second subunit of the Fc domain additionally the threonine residue at position 366 is replaced with a serine residue (T366S) and the leucine residue at position 368 is replaced with an alanine residue (L368A) (numberings according to Kabat EU index).

In yet further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In specific aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function. In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In some aspects, the effector function is one or more selected from the group of CDC, antibody-dependent cell-mediated cytotoxicity (ADCC), antibody-dependent cellular phagocytosis (ADCP), and cytokine secretion. In preferred aspects, the effector function is ADCC. In some aspects, the Fc domain domain exhibits substantially similar binding affinity to neonatal Fc receptor (FcRn), as compared to a native IgG1 Fc domain domain.

In some aspects, the Fc domain comprises amino acid substitutions at positions P329, L234 and L235 (numberings according to Kabat EU index). In more specific aspects, the Fc domain comprises the amino acid mutations L234A, L235A and P329G (“P329G LALA”, “PGLALA” or “LALAPG”). Specifically, in particular aspects, each of the first and the second subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In some such aspects, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

### 4. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table A under the heading of “preferred substitutions”. More substantial changes are provided in Table A under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. HCDR3 and LCDR3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region (see, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and γ₄₃₆ are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are 1253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region (see, e.g. Dall'Acqua et al. (2006) J Biol Chem 281, 23514-23524; WO2002/60919). In certain aspects, the antibody variant comprises an Fc region with amino acid substitutions at positions 428 and 434 (EU numbering of residues). In one aspect, the substitutions are M428L and N434S (see e.g. Zalevsky et al. (2010) Nat Biotech 28, 157-159; WO2009/086320). Further amino acid substitutions to which increase FcRn binding can be found in e.g. Maeda et al. (2017) MABS 9, 844-853 and WO2016/125493.

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In preferred aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. No. 7,521,541, 8,30,930, 7,855,275, 9,000,130, or WO 2016040856.

### e) Antibody Derivatives

In certain aspects, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 5. Immunoconjugates

The invention also provides immunoconjugates comprising an anti-CEACAM5 antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody of the invention conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody of the invention conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, MRI), such as I¹²³, I¹³¹, In¹¹¹, F¹⁹, C¹³, N¹⁵, O¹⁷, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available (e.g., from Pierce Biotechnology, Inc., Rockford, IL., USA).

### B. Polynucleotides

The invention further provides an isolated polynucleotide encoding an antibody of the invention. Said isolated polynucleotide may be a single polynucleotide or a plurality of polynucleotides.

The polynucleotides encoding antibodies of the invention may be expressed as a single polynucleotide that encodes the entire antibody or as multiple (e.g., two or more) polynucleotides that are co-expressed. Polypeptides encoded by polynucleotides that are co-expressed may associate through, e.g., disulfide bonds or other means to form a functional antibody. For example, the light chain portion of an antibody may be encoded by a separate polynucleotide from the portion of the antibody comprising the heavy chain of the antibody. When co-expressed, the heavy chain polypeptides will associate with the light chain polypeptides to form the antibody. In another example, the portion of the antibody comprising one of the two Fc domain subunits and optionally (part of) one or more Fab molecules could be encoded by a separate polynucleotide from the portion of the antibody comprising the other of the two Fc domain subunits and optionally (part of) a Fab molecule. When co-expressed, the Fc domain subunits will associate to form the Fc domain.

In some aspects, the isolated polynucleotide encodes the entire antibody molecule according to the invention as described herein. In other aspects, the isolated polynucleotide encodes a polypeptide comprised in the antibody according to the invention as described herein.

In certain aspects, the polynucleotide or nucleic acid is DNA. In other aspects, a polynucleotide of the present invention is RNA, for example, in the form of messenger RNA (mRNA). RNA of the present invention may be single stranded or double stranded.

### C. Recombinant Methods

Antibodies of the invention may be obtained, for example, by solid-state peptide synthesis (e.g. Merrifield solid phase synthesis) or recombinant production. For recombinant production one or more polynucleotide encoding the antibody, e.g., as described above, is isolated and inserted into one or more vectors for further cloning and/or expression in a host cell. Such polynucleotide may be readily isolated and sequenced using conventional procedures. In some aspects a vector, particularly an expression vector, comprising the polynucleotide (i.a. a single polynucleotide or a plurality of polynucleotides) of the invention is provided. Methods which are well known to those skilled in the art can be used to construct expression vectors containing the coding sequence of an antibody along with appropriate transcriptional/translational control signals. These methods include in vitro recombinant DNA techniques, synthetic techniques and in vivo recombination/genetic recombination. See, for example, the techniques described in Maniatis et al., Molecular Cloning: A Laboratory Manual, Cold Spring Harbor Laboratory, N.Y. (1989); and Ausubel et al., Current Protocols in Molecular Biology, Greene Publishing Associates and Wiley Interscience, N.Y (1989). The expression vector can be part of a plasmid, virus, or may be a nucleic acid fragment. The expression vector includes an expression cassette into which the polynucleotide encoding the antibody (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible by tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody is desired, DNA encoding a signal sequence may be placed upstream of the nucleic acid encoding an antibody of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain aspects, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse B-glucuronidase.

DNA encoding a short protein sequence that could be used to facilitate later purification (e.g. a histidine tag) or assist in labeling the antibody may be included within or at the ends of the antibody (fragment) encoding polynucleotide.

In a further aspect, a host cell comprising a polynucleotide (i.e. a single polynucleotide or a plurality of polynucleotides) of the invention is provided. In certain aspects a host cell comprising a vector of the invention is provided. The polynucleotides and vectors may incorporate any of the features, singly or in combination, described herein in relation to polynucleotides and vectors, respectively. In some such aspects, a host cell comprises (e.g. has been transformed or transfected with) one or more vector comprising one or more polynucleotide that encodes (part of) an antibody of the invention. As used herein, the term “host cell” refers to any kind of cellular system which can be engineered to generate the antibody of the invention or fragments thereof. Host cells suitable for replicating and for supporting expression of antibodies are well known in the art. Such cells may be transfected or transduced as appropriate with the particular expression vector and large quantities of vector containing cells can be grown for seeding large scale fermenters to obtain sufficient quantities of the antibody for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In some aspects, the host cell is a eukaryotic cell, particularly a mammalian cell, such as a Chinese Hamster Ovary (CHO) cell, a human embryonic kidney (HEK) cell or a lymphoid cell (e.g., YO, NS0, Sp20 cell). In some aspects, the host cell is an isolated host cell. In some aspects, the host cell is not a cell within a human body.

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one aspect, a method of producing an antibody according to the invention is provided, wherein the method comprises culturing a host cell comprising a polynucleotide encoding the antibody, as provided herein, under conditions suitable for expression of the antibody, and optionally recovering the antibody from the host cell (or host cell culture medium).

The components of the (multispecific) antibody of the invention may be genetically fused to each other. The (multispecific) antibody can be designed such that its components are fused directly to each other or indirectly through a linker sequence. The composition and length of the linker may be determined in accordance with methods well known in the art and may be tested for efficacy. Examples of linker sequences between different components of (multispecific) antibodies are provided herein. Additional sequences may also be included to incorporate a cleavage site to separate the individual components of the fusion if desired, for example an endopeptidase recognition sequence.

Antibodies prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification, an antibody, ligand, receptor or antigen can be used to which the antibody binds. For example, for affinity chromatography purification of antibodies of the invention, a matrix with protein A or protein G may be used. Sequential Protein A or G affinity chromatography and size exclusion chromatography can be used to isolate an antibody essentially as described in the Examples. The purity of the antibody can be determined by any of a variety of well-known analytical methods including gel electrophoresis, high pressure liquid chromatography, and the like.

### D. Compositions, Formulations, and Routes of Administration

In a further aspect, the invention provides pharmaceutical compositions comprising any of the antibodies provided herein, e.g., for use in any of the below therapeutic methods. In some aspects, a pharmaceutical composition comprises an antibody according to the invention and a pharmaceutically acceptable carrier. In further aspects, a pharmaceutical composition comprises an antibody according to the invention and at least one additional therapeutic agent, e.g., as described below.

Further provided is a method of producing an antibody of the invention in a form suitable for administration in vivo, the method comprising (a) obtaining an antibody according to the invention, and (b) formulating the antibody with at least one pharmaceutically acceptable carrier, whereby a preparation of antibody is formulated for administration in vivo.

Pharmaceutical compositions of the present invention comprise an effective amount of antibody dissolved or dispersed in a pharmaceutically acceptable carrier. The phrase “pharmaceutically acceptable” refers to molecular entities and compositions that are generally nontoxic to recipients at the dosages and concentrations employed, i.e. do not produce an adverse, allergic or other untoward reaction when administered to an animal, such as, for example, a human, as appropriate. The preparation of a pharmaceutical composition that contains an antibody and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

An antibody of the invention (and any additional therapeutic agent) can be administered by any suitable means, including parenteral, intrapulmonary, and intranasal, and, if desired for local treatment, intralesional administration. Parenteral administration includes intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the antibodies of the invention may be formulated in aqueous solutions, particularly in physiologically compatible buffers such as Hanks' solution, Ringer's solution, or physiological saline buffer. The solution may contain formulatory agents such as suspending, stabilizing and/or dispersing agents. Alternatively, the antibodies may be in powder form for constitution with a suitable vehicle, e.g., sterile pyrogen-free water, before use. Sterile injectable solutions are prepared by incorporating the antibodies of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable carriers include, but are not limited to: buffers such as histidine, phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Pharmaceutical compositions comprising the antibodies of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The antibodies may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### E. Therapeutic Methods and Compositions

Any of the antibodies provided herein may be used in therapeutic methods. Antibodies of the invention may be used as immunotherapeutic agents, for example in the treatment of cancers, in particular cancers characterized by the expression of CEACAM5, such as colorectal cancer.

For use in therapeutic methods, antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some aspects, antibodies of the invention for use as a medicament are provided. In further aspects, antibodies of the invention for use in treating a disease are provided. In certain aspects, antibodies of the invention for use in a method of treatment are provided. In some aspects, the invention provides an antibody of the invention for use in the treatment of a disease in an individual in need thereof. In certain aspects, the invention provides an antibody for use in a method of treating an individual having a disease comprising administering to the individual an effective amount of the antibody. In certain aspects, the disease is a proliferative disorder. In certain aspects, the disease is cancer, particularly a CEACAM5-expressing cancer, for example colorectal cancer. In certain aspects, the method further comprises administering to the individual an effective amount of at least one additional therapeutic agent, e.g., an anti-cancer agent if the disease to be treated is cancer. An “individual” according to any of the above aspects may be a mammal, preferably a human.

In a further aspect, the invention provides for the use of an antibody of the invention in the manufacture or preparation of a medicament. In some aspects, the medicament is for the treatment of a disease in an individual in need thereof. In a further aspect, the medicament is for use in a method of treating a disease comprising administering to an individual having the disease an effective amount of the medicament. In certain aspects, the disease is a proliferative disorder. In certain aspects, the disease is cancer, particularly a CEACAM5-expressing cancer, for example colorectal cancer. In some aspects, the method further comprises administering to the individual an effective amount of at least one additional therapeutic agent, e.g., an anti-cancer agent if the disease to be treated is cancer. An “individual” according to any of the above aspects may be a mammal, preferably a human.

In a further aspect, the invention provides a medicament (adapted) for the treatment of a disease, comprising the antibody of the invention. In some aspects the medicament is (adapted) for the treatment of a disease in an individual in need thereof. In further aspects, the medicament is (adapted) for use in a method of treating a disease comprising administering to an individual having the disease an effective amount of the medicament. In certain aspects, the disease is a proliferative disorder. In certain aspects, the disease is cancer, particularly a CEACAM5-expressing cancer, for example colorectal cancer. In some aspects, the treatment or method of treating further comprises administering to the individual an effective amount of at least one additional therapeutic agent, e.g., an anti-cancer agent if the disease to be treated is cancer. An “individual” according to any of the above aspects may be a mammal, preferably a human.

In a further aspect, the invention provides a method for treating a disease. In some aspects, the method comprises administering to an individual having such disease an effective amount of an antibody of the invention. In some aspects, a composition is administered to said individual, comprising the antibody of the invention in a pharmaceutically acceptable form. In certain aspects, the disease is a proliferative disorder. In certain aspects, the disease is cancer, particularly a CEACAM5-expressing cancer, for example colorectal cancer. In certain aspects, the method further comprises administering to the individual an effective amount of at least one additional therapeutic agent, e.g., an anti-cancer agent if the disease to be treated is cancer. An “individual” according to any of the above aspects may be a mammal, preferably a human.

In certain aspects, the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain aspects, the cancer is a cancer expressing CEACAM5. In some aspects, the cancer is selected from the group consisting of colorectal cancer, gastric cancer, pancreatic cancer lung cancer (e.g. non-small cell lung cancer (NSCLC)) and breast cancer. A skilled artisan readily recognizes that in many cases the antibody may not provide a cure but may only provide partial benefit. In some aspects, a physiological change having some benefit is also considered therapeutically beneficial. Thus, in some aspects, an amount of antibody that provides a physiological change is considered an “effective amount”. The subject, patient, or individual in need of treatment is typically a mammal, more specifically a human.

In some aspects, an effective amount of an antibody of the invention is administered to an individual for the treatment of disease.

For the prevention or treatment of disease, the appropriate dosage of an antibody of the invention (when used alone or in combination with one or more other additional therapeutic agents) will depend on the type of disease to be treated, the route of administration, the body weight of the patient, the type of antibody, the severity and course of the disease, whether the antibody is administered for preventive or therapeutic purposes, previous or concurrent therapeutic interventions, the patient's clinical history and response to the antibody, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. The progress of this therapy is easily monitored by conventional techniques and assays.

The antibodies of the invention will generally be used in an amount effective to achieve the intended purpose. For use to treat or prevent a disease condition, the antibodies of the invention, or pharmaceutical compositions thereof, are administered or applied in an effective amount.

For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art.

Dosage amount and interval may be adjusted individually to provide plasma levels of the antibodies which are sufficient to maintain therapeutic effect. Therapeutically effective plasma levels may be achieved by administering multiple doses each day. Levels in plasma may be measured, for example, by HPLC.

An effective dose of the antibodies of the invention will generally provide therapeutic benefit without causing substantial toxicity. Toxicity and therapeutic efficacy of an antibody can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Antibodies that exhibit large therapeutic indices are preferred. In some aspects, the antibody according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with antibodies of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The antibodies of the invention may be administered in combination with one or more other agents in therapy. For instance, an antibody of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular disease being treated, preferably those with complementary activities that do not adversely affect each other. In certain aspects, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In certain aspects, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of antibody used, the type of disorder or treatment, and other factors discussed above. The antibodies are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the antibody of the invention can occur prior to, simultaneously, and/or following, administration of the additional therapeutic agent and/or adjuvant. Antibodies of the invention may also be used in combination with radiation therapy.

### F. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### G. Methods and Compositions for Diagnostics and Detection

In certain aspects, any of the antibodies provided herein is useful for detecting the presence of CEACAM5 in a biological sample. The term “detecting” as used herein encompasses quantitative or qualitative detection. In certain aspects, a biological sample comprises a cell or tissue, such as tumor tissue.

In one aspect, an antibody according to the invention for use in a method of diagnosis or detection is provided. In a further aspect, a method of detecting the presence of CEACAM5 in a biological sample is provided. In certain aspects, the method comprises contacting the biological sample with an antibody of the present invention under conditions permissive for binding of the antibody to CEACAM5, and detecting whether a complex is formed between the antibody and CEACAM5. Such method may be an in vitro or in vivo method. In some aspects, an antibody of the invention is used to select subjects eligible for therapy with an antibody that binds CEACAM5, e.g. where CEACAM5 is a biomarker for selection of patients.

Exemplary disorders that may be diagnosed using an antibody of the invention include cancer, particularly CEACAM5 expressing cancers, for example colorectal cancer.

In certain aspects, an antibody according to the present invention is provided, wherein the antibody is labelled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, B-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### III. Sequences

| | | SEQ ID |
|---|---|---|
| Name | Sequence | NO |
| T84.66 (P1AH2337) HCDR1 | DTYMH | 1 |
| T84.66 (P1AH2337) HCDR2 | RIDPANGNSKYVPKFQG | 2 |
| T84.66 (P1AH2337) HCDR3 | FGYYVSDYAMAY | 3 |
| T84.66 (P1AH2337) VH | EVQLQQSGAELVEPGASVKLSCTASGENIKDTYMHWVKQRPEQGL | 4 |
| | EWIGRIDPANGNSKYVPKFQGKATITADTSSNTAYLQLTSLTSED | |
| | TAVYYCAPFGYYVSDYAMAYWGQGTSVTVSS | |
| T84.66 (P1AH2337) LCDR1 | RAGESVDIFGVGFLH | 5 |
| T84.66 (P1AH2337) LCDR2 | RASNLES | 6 |
| T84.66 (P1AH2337) LCDR3 | QQTNEDPYT | 7 |
| T84.66 (P1AH2337) VL | DIVLTQSPASLAVSLGQRATMSCRAGESVDIFGVGFLHWYQQKPG | 8 |
| | QPPKLLIYRASNLESGIPVRFSGTGSRTDFTLIIDPVEADDVATY | |
| | YCQQTNEDPYTFGGGTKLEIK | |
| T84.66-LCHA (P1AH2338) | DTYMH | 1 |
| HCDR1 | | |
| T84.66-LCHA (P1AH2338) | RIDPANGNSKYVPKFQG | 2 |
| HCDR2 | | |
| T84.66-LCHA (P1AH2338) | FGYYVSDYAMAY | 3 |
| HCDR3 | | |
| T84.66-LCHA (P1AH2338) | QVQLVQSGAEVKKPGSSVKVSCKASGFNIKDTYMHWVRQAPGQGL | 9 |
| VH | EWMGRIDPANGNSKYVPKFQGRVTITADTSTSTAYMELSSLRSED | |
| | TAVYYCAPFGYYVSDYAMAYWGQGTLVTVSS | |
| T84.66-LCHA (P1AH2338) | RAGESVDIFGVGELH | 5 |
| LCDR1 | | |
| T84.66-LCHA (P1AH2338) | RASNRAT | 10 |
| LCDR2 | | |
| T84.66-LCHA (P1AH2338) | QQTNEDPYT | 7 |
| LCDR3 | | |
| T84.66-LCHA (P1AH2338) | EIVLTQSPATLSLSPGERATLSCRAGESVDIFGVGFLHWYQQKPG | 11 |
| VL | QAPRLLIYRASNRATGIPARFSGSGSGTDFTLTISSLEPEDFAVY | |
| | YCQQTNEDPYTFGQGTKLEIK | |
| P1AH2349/P1AI5014/ | DTYMH | 1 |
| P1AI5015/P1AI5016/ | | |
| P1AI5017/P1AI5018/ | | |
| P1AI5031 HCDR1 | | |
| P1AH2349/P1AI5014/ | RIDPANGNSKYADSVKG | 12 |
| P1AI5015/P1AI5016/ | | |
| P1AI5017/P1AI5018/ | | |
| P1AI5031 HCDR2 | | |
| P1AH2349/P1AI5014/ | FGYYVSDYAMAY | 3 |
| P1AI5015/P1AI5016/ | | |
| P1AI5017/P1AI5018/ | | |
| P1AI5031 HCDR3 | | |
| P1AH2349/P1AI5014/ | EVQLVESGGGLVQPGGSLRLSCAASGENIKDTYMHWVRQAPGKGL | 13 |
| P1AI5015/P1AI5016/ | EWVGRIDPANGNSKYADSVKGRFTISADTSKNTAYLQMNSLRAED | |
| P1AI5017/P1AI5018/ | TAVYYCAPFGYYVSDYAMAYWGQGTTVTVSS | |
| P1AI5031 VH | | |
| P1AI5014 LCDR1 | RAGESVDIFGVGFLH | 5 |
| P1AI5014 LCDR2 | RGSNRAT | 14 |
| P1AI5014 LCDR3 | QQTNEDPYT | 7 |
| P1AI5014 VL | EIVLTQSPATLSLSPGERATLSCRAGESVDIFGVGELHWYQQKPG | 15 |
| | QAPRLLIYRGSNRATGIPARFSGSGSGTDFTLTISSLEPEDFAVY | |
| | YCQQTNEDPYTFGQGTKLEIK | |
| P1AI5015 LCDR1 | RAGESVDIFGVGELH | 5 |
| P1AI5015 LCDR2 | RGSNRAT | 14 |
| P1AI5015 LCDR3 | QQTYEYPYT | 16 |
| P1AI5015 VL | EIVLTQSPATLSLSPGERATLSCRAGESVDIFGVGELHWYQQKPG | 17 |
| | QAPRLLIYRGSNRATGIPARFSGSGSGTDFTLTISSLEPEDFAVY | |
| | YCQQTYEYPYTFGQGTKLEIK | |
| P1AI5016 LCDR1 | RAGESVDIFGVGELH | 5 |
| P1AI5016 LCDR2 | RGSNRAT | 14 |
| P1AI5016 LCDR3 | QQTVEYPYT | 18 |
| P1AI5016 VL | EIVLTQSPATLSLSPGERATLSCRAGESVDIFGVGELHWYQQKPG | 19 |
| | QAPRLLIYRGSNRATGIPARFSGSGSGTDFTLTISSLEPEDFAVY | |
| | YCQQTVEYPYTFGQGTKLEIK | |
| P1AI5017 LCDR1 | RAGESVDIFGVGFLH | 5 |
| P1AI5017 LCDR2 | RGSNRAT | 14 |
| P1AI5017 LCDR3 | QQTSEYPYT | 20 |
| P1AI5017 VL | EIVLTQSPATLSLSPGERATLSCRAGESVDIFGVGFLHWYQQKPG | 21 |
| | QAPRLLIYRGSNRATGIPARFSGSGSGTDFTLTISSLEPEDFAVY | |
| | YCQQTSEYPYTFGQGTKLEIK | |
| P1AI5018 LCDR1 | RAGESVDIFGVGELH | 5 |
| P1AI5018 LCDR2 | RGSNRAT | 14 |
| P1AI5018 LCDR3 | QQTTEYPYT | 22 |
| P1AI5018 VL | EIVLTQSPATLSLSPGERATLSCRAGESVDIFGVGFLHWYQQKPG | 23 |
| | QAPRLLIYRGSNRATGIPARFSGSGSGTDFTLTISSLEPEDFAVY | |
| | YCQQTTEYPYTFGQGTKLEIK | |
| P1AI5031 LCDR1 | RAGESVDIFGVGFLH | 5 |
| P1AI5031 LCDR2 | RGSNRMT | 24 |
| P1AI5031 LCDR3 | QQTNEDPYT | 7 |
| P1AI5031 VL | EIVLTQSPATLSLSPGERATLSCRAGESVDIFGVGELHWYQQKPG | 25 |
| | QAPRLLIYRGSNRMTGIPARFSGSGSGTDFTLTISSLEPEDFAVY | |
| | YCQQTNEDPYTFGQGTKLEIK | |
| LCDR2 (consensus) | RX₁SNRX₂T | 26 |
| | X₁ = A or G; X₂ = A or M | |
| LCDR2 (consensus) | RGSNRX₁T | 27 |
| | X₁ = A or M | |
| LCDR3 (consensus) | QQTX₁EX₂PYT | 28 |
| | X₁ = N, Y, V, S or T; X₂ = D or Y | |
| VL (consensus) | EIVLTQSPATLSLSPGERATLSCRAGESVDIFGVGELHWYQQKPG | 29 |
| | QAPRLLIYRX₁SNRX₂TGIPARFSGSGSGTDFTLTISSLEPEDFA | |
| | VYYCQQTX₃EX₄PYTFGQGTKLEIK | |
| | X₁ = A or G; X₂ = A or M; X₃ = N, Y, V, S or | |
| | T; X₄ = D or Y | |
| VL (consensus) | EIVLTQSPATLSLSPGERATLSCRAGESVDIFGVGFLHWYQQKPG | 30 |
| | QAPRLLIYRGSNRX₁TGIPARFSGSGSGTDFTLTISSLEPEDFAV | |
| | YYCQQTX₂EX₃PYTFGQGTKLEIK | |
| | X₁ = A or M; X₂ = N, Y, V, S or T; X₃ = D or Y | |
| H-FR1 | EVQLVESGGGLVQPGGSLRLSCAASGENIK | 31 |
| H-FR2 | WVRQAPGKGLEWVG | 32 |
| H-FR3 | RFTISADTSKNTAYLQMNSLRAEDTAVYYCAP | 33 |
| H-FR4 | WGQGTTVTVSS | 34 |
| L-FR1 | EIVLTQSPATLSLSPGERATLSC | 35 |
| L-FR2 | WYQQKPGQAPRLLIY | 36 |
| L-FR3 | GIPARFSGSGSGTDFTLTISSLEPEDFAVYYC | 37 |
| L-FR4 | FGQGTKLEIK | 38 |
| hIgG1 Fc region | DKTHTCPPCPAPELLGGPSVFLFPPKPKDTLMISRTPEVTCVVVD | 39 |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| Human kappa CL domain | RTVAAPSVFIFPPSDEQLKSGTASVVCLLNNFYPREAKVQWKVDN | 40 |
| | ALQSGNSQESVTEQDSKDSTYSLSSTLTLSKADYEKHKVYACEVT | |
| | HQGLSSPVTKSENRGEC | |
| Human lambda CL domain | QPKAAPSVTLFPPSSEELQANKATLVCLISDFYPGAVTVAWKADS | 41 |
| | SPVKAGVETTTPSKQSNNKYAASSYLSLTPEQWKSHRSYSCQVTH | |
| | EGSTVEKTVAPTECS | |
| Human IgG1 heavy chain | ASTKGPSVFPLAPSSKSTSGGTAALGCLVKDYFPEPVTVSWNSGA | 42 |
| constant region (CH1-CH2- | LTSGVHTFPAVLQSSGLYSLSSVVTVPSSSLGTQTYICNVNHKPS | |
| CH3) | NTKVDKKVEPKSCDKTHTCPPCPAPELLGGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSP | |
| P1AI5013 LCDR1 | RAGESVDHFGVGELH | 43 |
| P1AI5013 LCDR2 | RASNRAT | 10 |
| P1AI5013 LCDR3 | QQTNEDPYT | 7 |
| P1AI5013 VL | EIVLTQSPATLSLSPGERATLSCRAGESVDHFGVGELHWYQQKPG | 44 |
| | QAPRLLIYRASNRATGIPARFSGSGSGTDFTLTISSLEPEDFAVY | |
| | YCQQTNEDPYTFGQGTKLEIK | |
| CD3 HCDR1 | SYAMN | 45 |
| CD3 HCDR2 | RIRSKYNNYATYYADSVKG | 46 |
| CD3 HCDR3 | ASNFPASYVSYFAY | 47 |
| CD3 VH | EVQLLESGGGLVQPGGSLRLSCAASGFTFSSYAMNWVRQAPGKGL | 48 |
| | EWVSRIRSKYNNYATYYADSVKGRFTISRDDSKNTLYLQMNSLRA | |
| | EDTAVYYCVRASNFPASYVSYFAYWGQGTLVTVSS | |
| CD3 LCDR1 | GSSTGAVTTSNYAN | 49 |
| CD3 LCDR2 | GTNKRAP | 50 |
| CD3 LCDR3 | ALWYSNLWV | 51 |
| CD3 VL | QAVVTQEPSLTVSPGGTVTLTCGSSTGAVTTSNYANWVQEKPGQA | 52 |
| | FRGLIGGTNKRAPGTPARFSGSLLGGKAALTLSGAQPEDEAEYYC | |
| | ALWYSNLWVFGGGTKLTVL | |

### IV. Examples

The following are examples of methods and compositions of the invention. It is understood that various other aspects may be practiced, given the general description provided above.

### Example 1—Design of T84.66 Humanization Variants to Improve Expression Titers

The previously humanized version of the anti-CEACAM5 antibody T84.66, T84.66-LCHA (“humanized variant 1”/SEQ ID NOs 22 and 23 of WO 2017/055389; SEQ ID NOs 9 and 11 herein), is affected by low expression titers in common mammalian expression systems, limiting its use in particular in the context of novel, complex protein formats. A trend for rather low expression titers is also traceable for the parental, murine antibody T84.66, but to a lesser extent.

In order to obtain a humanized version of T84.66 with better expression properties, a number of variants of both VH and VL were designed. As the main driver for the low expression could not be clearly identified based on the available data, the variants were designed such as to address different potential liabilities. In general, the following approaches were pursued: Exchange of the human acceptor framework (rehumanization), substitution of solvent-exposed hydrophobic amino acids (removal of hydrophobic patches), substitution of solvent-exposed negatively charged amino acids (removal of acidic patches), and, for some positions in the variable region, substitution of infrequently occurring amino acids by more frequently occurring ones (when using human V region germlines as the reference set).

Furthermore, two VH and two VL variants were obtained by a similarity search over the full variable domain with BLASTp (Camacho et al. (2009) BMC Bioinformatics 10, 421) in the Observed Antibody Space (OAS) database (https://opig.stats.ox.ac.uk/webapps/oas/; dated 15 Sep. 2021), and one VH variant was generated by a Gated Recurrent Unit (GRU)-based generative model that was learned from the top 1000 BLASTp hits in OAS. For the GRU-generated set, only the most frequently generated sequences were taken. The CDRs of the parental T84.66 clones were grafted onto the identified frameworks from repertoire search and the GRU model. For the GRU-generated VH, two further residues downstream of the H-CDR2 were also grafted from the parental sequence to maintain physicochemical homology.

The acidic patch removal variants were designed based on published findings that antibody variable regions with negatively charged patches can be subject to the formation of inclusion bodies in the ER (Hasegawa et al. (2017) Cell Logist 7, e1361499, and Hasegawa et al. (2021) Biochim Biophys Acta Mol Cell Res 1868, 119078).

The antibodies that were designed are listed in Table 1. Note that the humanized T84.66-LCHA uses the human V region germlines IGHV1-3*01 and IGKV3-20*02. All VH variants were paired with the reference humanized VL, T84.66-LCHA_VL (bold). All VL variants were paired with the reference humanized VH, T84.66-LCHA_VH (bold). The singular exception is antibody P1AH2375, where both VH and VL are acidic patch removal variants.

TABLE 1
List of the antibodies that were produced for expression optimization

| ID | Name of VH | Name of VL | Description |
|---|---|---|---|
| P1AH2337 | T84.66_{—}VH | T84.66_{—}VL | Murine reference |
| P1AH2338 | T84.66-LCHA_{—}VH | T84.66-LCHA_{—}VL | Humanized reference |
| P1AH2339 | VH1a_HV1-69-2_1 | T84.66-LCHA_{—}VL | VH rehumanization |
| P1AH2340 | VH1b_HV1-69-2_1_fm | T84.66-LCHA_{—}VL | VH rehumanization |
| P1AH2341 | VH2a_HV1-46_1 | T84.66-LCHA_{—}VL | VH rehumanization |
| P1AH2342 | VH2b_HV1-46_1_fm | T84.66-LCHA_{—}VL | VH rehumanization |
| P1AH2343 | VH3a_HV1-3_1 | T84.66-LCHA_{—}VL | VH rehumanization |
| P1AH2344 | VH3b_HV1-3_1_fm | T84.66-LCHA_{—}VL | VH rehumanization |
| P1AH2345 | VH3c_HV1-3_1_bm | T84.66-LCHA_{—}VL | VH rehumanization |
| P1AH2346 | VH3d_HV1-3_1_bm_fm | T84.66-LCHA_{—}VL | VH rehumanization |
| P1AH2347 | VH4a_HV1-69_1 | T84.66-LCHA_{—}VL | VH rehumanization |
| P1AH2348 | VH4b_HV1-69_1_fm | T84.66-LCHA_{—}VL | VH rehumanization |
| P1AH2349 | VH5a_Herc-like* | T84.66-LCHA_{—}VL | VH rehumanization |
| P1AH2350 | T84.66-LCHA_VH_P94V | T84.66-LCHA_{—}VL | Proline substitution |
| P1AH2351 | T84.66-LCHA_VH_P94A | T84.66-LCHA_{—}VL | Proline substitution |
| P1AH2352 | T84.66-LCHA_VH_P94T | T84.66-LCHA_{—}VL | Proline substitution |
| P1AH2354 | T84.66-LCHA_VH_P94S | T84.66-LCHA_{—}VL | Proline substitution |
| P1AH2355 | T84.66-LCHA_VH_M100dK | T84.66-LCHA_{—}VL | Hydrophobic patch |
| P1AH2356 | T84.66-LCHA_VH_M100dQ | T84.66-LCHA_{—}VL | Hydrophobic patch |
| P1AH2357 | T84-HA-a_D31S | T84.66-LCHA_{—}VL | Acidic patch |
| P1AH2358 | T84-HA-b_D52Q | T84.66-LCHA_{—}VL | Acidic patch |
| P1AH2359 | T84-HA-c_E81Q | T84.66-LCHA_{—}VL | Acidic patch |
| P1AH2360 | T84-HA-d_D31S _E81Q | T84.66-LCHA_{—}VL | Acidic patch |
| P1AH2361 | T84-HA-e_K30T | T84.66-LCHA_{—}VL | Acidic patch |
| P1AH2362 | T84.66-LCHA_{—}VH | VL1a_KV1-39_01 | VL rehumanization |
| P1AH2363 | T84.66-LCHA_{—}VH | VL1a_KV1- | VL rehumanization |
| | | 39_01_Q55E_P95D | |
| P1AH2364 | T84.66-LCHA_{—}VH | T84.66-LCHA_VL_A51G | Hydrophobic patch |
| P1AH2365 | T84.66-LCHA_{—}VH | T84.66-LCHA_VL_I27dH | Hydrophobic patch |
| P1AH2366 | T84.66-LCHA_{—}VH | T84.66-LCHA_VL_F28Y | Hydrophobic patch |
| P1AH2367 | T84.66-LCHA_{—}VH | T84.66-LCHA_VL_F28S | Hydrophobic patch |
| P1AH2368 | T84.66-LCHA_{—}VH | T84.66-LCHA_VL_IF_SY | Hydrophobic patch |
| P1AH2369 | T84.66-LCHA_{—}VH | T84.66- | Hydrophobic patch |
| | | LCHA_VL_VG_NS | |
| P1AH2370 | T84.66-LCHA_{—}VH | T84-LC-a_D27cS | Acidic patch |
| P1AH2371 | T84.66-LCHA_{—}VH | T84-LC-b_LC_plus_LE | Acidic patch |
| P1AH2373 | T84.66-LCHA_{—}VH | T84-LC-d_D94N | Acidic patch |
| P1AH2374 | T84.66-LCHA_{—}VH | T84-LC-e_acidic_patch | Acidic patch |
| P1AH2375 | T84-HA-d_D31S_E81Q | T84-LC-e_acidic_patch | Acidic patch |
| P1AH2954 | OAS_VH_1 | T84.66-LCHA_{—}VL | Repertoire search |
| P1AH2955 | OAS_VH_2 | T84.66-LCHA_{—}VL | Repertoire search |
| P1AH2956 | GRU_VH_1 | T84.66-LCHA_{—}VL | Generative model |
| P1AH2957 | T84.66-LCHA_{—}VH | OAS_VL_1 | Repertoire search |
| P1AH2958 | T84.66-LCHA_{—}VH | OAS_VL_2 | Repertoire search |

*VH variant VH5a_Herc-like uses the human germline IGHV3-66*01 with a number of framework mutations adopted from the VH region of trastuzumab.

### Example 2—Recombinant Production of the T84.66 Humanization Variants

The T84.66 humanization variants of Example 1 were produced in human IgG₁ format as described in the following.

### Gene Synthesis

Desired gene segments were prepared by chemical synthesis and the synthesized gene fragments were cloned into a suitable vector for expression in HEK293 and Expi293 cells by Twist Bioscience (San Francisco, USA).

### Expression in Mammalian Cells

Antibody production was performed by transient co-transfection of single expression cassette plasmids in HEK293 cells cultivated in F17 Medium (Invitrogen, Carlsbad, CA, USA) or Expi293 cells in Expi293 Expression medium (Thermo Scientific, Waltham, MA, USA), respectively. Transfection was carried out as specified in the manufacturer's instructions with a plasmid ratio of heavy chain (HC):light chain (LC) expression plasmids of 1:1 in case of symmetric standard IgG1 format (or with a plasmid ratio of HC1:HC2:LC expression plasmids of 1:1:1 in case of asymmetric “2+1” formats, i.e. the TCBs used in Example 6). Cell culture supernatants were harvested seven days after transfection. Supernatants were stored at reduced temperature (e.g. −20° C.).

### Quantification of Protein Titer

The protein titer of supernatant samples was determined by affinity chromatography using a POROS A 20 μm column, 2.1×30 mm (Life Technologies, Carlsbad, CA, USA) on a high performance liquid chromatography system (Ultimate 3000 HPLC system, Thermo Scientific, Waltham, MA, USA). The supernatant was loaded onto the column equilibrated with 0.2 M Na₂HPO₄, pH 7.4, followed by elution with 0.1 M citric acid, 0.2 M NaCl, pH 2.5. Titers were quantified by measuring absorption at 280 nm, and subsequently calculating the protein concentration by comparing the elution peak area (under the curve) of the analyte with a reference standard curve.

Purification of Modified Antibody from Mammalian Culture Supernatant

Antibodies in the culture supernatant were captured by Protein A affinity chromatography using MabSelectSuRe-Sepharose (Cytiva, Marlborough, MA, USA) on a liquid handling system (Tecan, Mannedorf, Switzerland), equipped with columns from Repligen (Waltham, MA, USA). Columns were equilibrated with PBS buffer, pH 7.4. Unbound protein was removed by washing with equilibration buffer. The antibodies were eluted with 25 mM citrate, pH 3.0. The eluted antibody fractions were neutralized with 1.5 M Tris, pH 7.5 and the concentration was determined by measuring the optical density (OD) at 280 nm. Size exclusion chromatography using a Superdex 200™ column (GE Healthcare, Chicago, IL, USA) in 20 mM histidine, 140 mM NaCl, pH 6.0 was performed as second purification step. Purified antibodies were stored at −80° C.

TABLE 2
Yield and composition as main peak CE-SDS of the purified T84.66
humanization variants. In bold, parental clones and variants
with higher yield compared to parental (T84.66-LCHA)

| | prepSEC yield | CE-SDS monomer |
|---|---|---|
| Variant | [μg] | peak [%] |
| P1AH2337 (T84.66) | 636.1 | 98.3 |
| P1AH2338 (T84.66-LCHA) | 183.4 | 100.0 |
| P1AH2339 | 74.5 | 100.0 |
| P1AH2340 | 335.8 | 100.0 |
| P1AH2341 | 60.0 | 100.0 |
| P1AH2342 | 629.1 | 100.0 |
| P1AH2343 | 0.0 | 100.0 |
| P1AH2344 | 50.3 | 100.0 |
| P1AH2345 | 16.6 | 100.0 |
| P1AH2346 | 378.0 | 100.0 |
| P1AH2347 | 59.3 | 100.0 |
| P1AH2348 | 757.0 | 100.0 |
| P1AH2349 | 1341.3 | 99.4 |
| P1AH2350 | 1283.3 | 100.0 |
| P1AH2351 | 1494.0 | 100.0 |
| P1AH2352 | 1615.2 | 98.7 |
| P1AH2354 | 1615.2 | 98.8 |
| P1AH2355 | 55.9 | 100.0 |
| P1AH2356 | 0.0 | 100.0 |
| P1AH2357 | 120.0 | 100.0 |
| P1AH2358 | 100.0 | 100.0 |
| P1AH2359 | 160.0 | 100.0 |
| P1AH2360 | 59.7 | 100.0 |
| P1AH2361 | 1368.8 | 100.0 |
| P1AH2362 | 246.6 | 100.0 |
| P1AH2363 | 49.7 | 100.0 |
| P1AH2364 | 490.5 | 100.0 |
| P1AH2365 | 465.5 | 100.0 |
| P1AH2366 | 259.4 | 100.0 |
| P1AH2367 | 250.3 | 100.0 |
| P1AH2368 | 291.9 | 100.0 |
| P1AH2369 | 355.2 | 100.0 |
| P1AH2370 | 254.1 | 100.0 |
| P1AH2371 | 1458.0 | 97.6 |
| P1AH2373 | 39.5 | 100.0 |
| P1AH2374 | 0.0 | n.d. |
| P1AH2375 | 12.1 | n.d. |
| P1AH2954 | 1198.5 | 100.0 |
| P1AH2955 | 0.0 | n.d. |
| P1AH2956 | 574.4 | 95.3 |
| P1AH2957 | 0.0 | n.d. |
| P1AH2958 | 73.6 | 100.0 |

The sequence optimization of the T84.66 humanization variants led to up to 9-fold increased yield as compared to the parental humanized T84.66-LCHA. While also variants not expressing, or expressing at lower yield than the humanized T84.66-LCHA were observed during the optimization process, in particular mutations at VH-position Pro94 seem to significantly increase the yield. Other approaches of re-humanization, and removal of hydrophobic or acidic patches at different positions also led to an increased yield.

### Example 3—Binding of the T84.66 Humanization Variants to Cells

The purified T84.66 humanization variants (from Example 2) were tested for binding to human CEACAM5 expressing cells (MKN-45) by FACS, and EC50 values were calculated. The cellular binding assay was performed as follows.

### Buffer and Cell Culture Medium:

DPBS pH 7.4 (PAN BIOTECH #P04-36500) containing 0.1% BSA was used as assay buffer. Cells were cultured in RPMI 1640 (Gibco #52400) medium with 20% heat inactivated FBS (Gibco #1050064) and 1×L-glutamine (Gibco #25030-081) for 3 passages.

### Method

10 μl of a 1×10⁴ MKN-45 (DSMZ ACC409) cell suspension in assay buffer was seeded in all Wells of a 384 well polypropylene plate (Weidmann #23490-101). Then 10 μl of antibodies diluted in assay buffer (double concentrated), at a start concentration of 100 μg/ml, serially diluted 1:4 were added to the plate. After 90 minutes incubation at 4° C., 60 μl assay buffer was added followed by a centrifuge step (330×g, 5 min) and an aspirating step (BioTek washer 405H) where 60 μl of supernatant were discarded. 20 μl of detection antibody (Alpaca anti rabbit IgG Chromotek #srbAF647-1) diluted in assay buffer (double concentrated) to a final concentration of 0.5 μg/ml was added to all Wells followed by 1 hour incubation time at room temperature. The cells were then subjected to two washing steps with 40 μl and 60 μl assay buffer as described above and then resuspended in 20 μl assay buffer. Cell binding was measured on an iQue Screener Plus high throughput FACS device from Sartorius/Intellicyt.

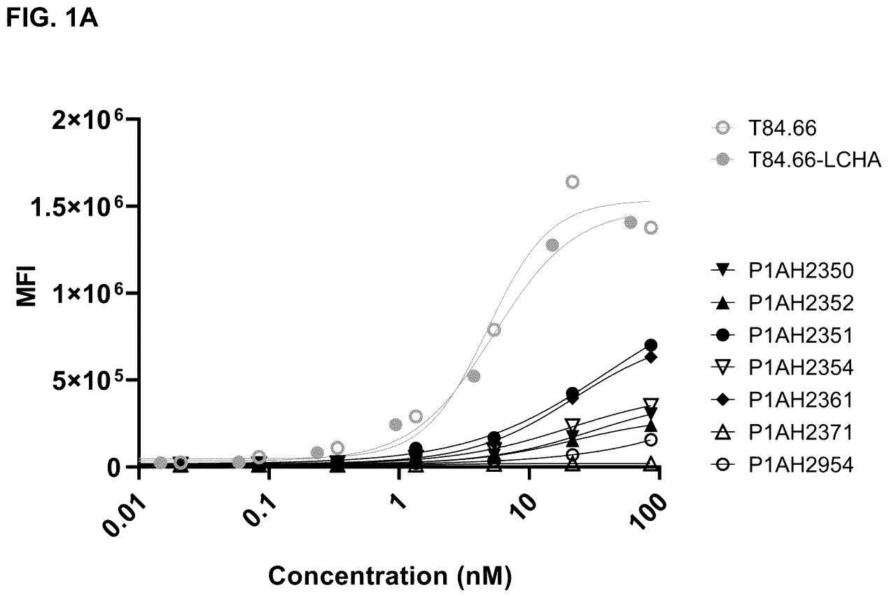

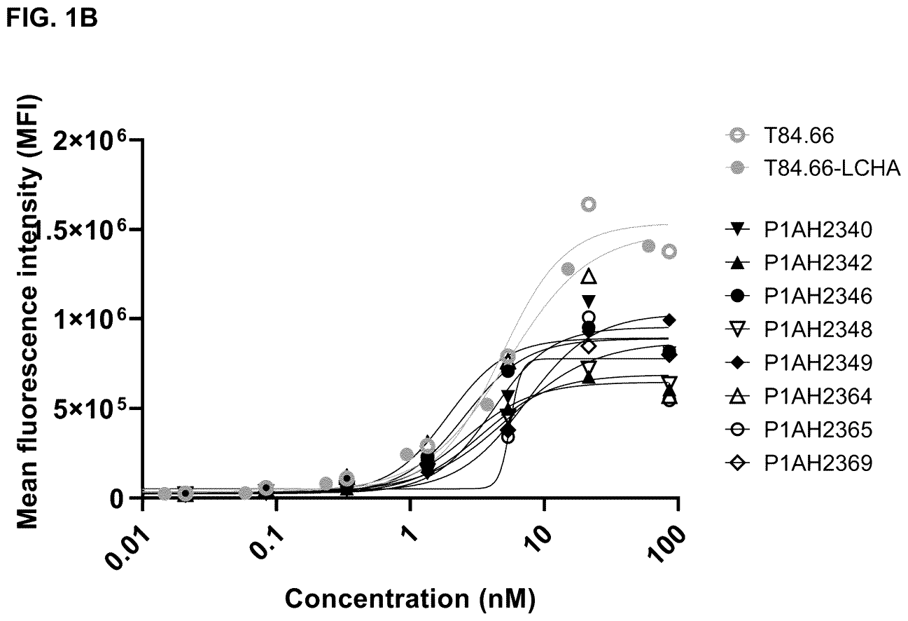

Results are shown in FIG. 1 and Table 3. Selected variants with increased yield as compared to T84.66-LCHA and sufficient CEACAM5 binding were characterized further as described below.

TABLE 3
EC50 from FACS binding study with MKN-45 CEACAM5 expressing
cells. In bold, T84.66 humanization variants with increased
yield as compared to T84.66-LCHA, that were further tested.

| Variant | EC50 [nM] |
|---|---|
| P1AH2339 | na |
| P1AH2340 | 4.1 |
| P1AH2341 | 21.4 |
| P1AH2342 | 2.6 |
| P1AH2344 | 30.7 |
| P1AH2345 | na |
| P1AH2346 | 2.5 |
| P1AH2347 | 3.1 |
| P1AH2348 | 3.4 |
| P1AH2349 | 7.2 |
| P1AH2350 | 30.1 |
| P1AH2351 | 39.1 |
| P1AH2352 | 21.9 |
| P1AH2354 | 20.4 |
| P1AH2355 | na |
| P1AH2357 | 2.3 |
| P1AH2358 | na |
| P1AH2359 | 12.9 |
| P1AH2360 | na |
| P1AH2361 | 21.9 |
| P1AH2362 | 4.7 |
| P1AH2363 | 15.5 |
| P1AH2364 | 1.9 |
| P1AH2365 | na |
| P1AH2366 | 2.7 |
| P1AH2367 | na |
| P1AH2368 | 2.5 |
| P1AH2369 | 5.5 |
| P1AH2370 | 3.1 |
| P1AH2371 | 3.2 |
| P1AH2373 | 2.5 |
| P1AH2954 | na |
| P1AH2956 | 12.1 |
| P1AH2958 | 32.2 |
| T84.66 | 4.8 |
| T84.66-LCHA | 5.3 |

Selected T84.66 humanization variants with increased yield and sufficient binding to the target were further characterized in an additional binding study on CEACAM5 expressing LS180 cells. The cellular binding assays was performed as follows.

LS180 (ATCC CL-187) cells were collected using trypsin and viability was checked. Then 100 μl of re-suspended target cells were seeded into a U-bottom plate (100′000 cells/well). The antibodies were diluted in FACS buffer (1×PBS, 2% FBS, 5 mM EDTA pH 8.0, 0.05% NaN₃). The plates were centrifuged for 4 min at 400×g and supernatant was removed. 50 μl/well of the diluted (1/1000 in PBS) Live/Dead Near Infrared (NIR) stain (Invitrogen, #L34976) were added to the cells. The cells were incubated for 30 min at 4° C. After the incubation, cells were washed twice. Then 30 μl/well of the diluted antibodies were added to the cells. The cells were incubated for 30 min at 4° C. After the incubation, cells were washed twice and 30 μl/well of diluted (1/50 in FACS buffer) FITC anti-human Fc secondary antibody (Jackson Immuno Research, #109-096-098) was added to the cells. The cells were incubated for 30 min at 4° C. After the incubation, cells were washed twice by adding 150 μl/well of FACS buffer, by centrifugation for 4 min at 400×g and by removing the supernatant after each wash. Then cells were re-suspended in 150 μl/well of FACS buffer. Cell binding was measured on the BD LSRFortessa Flow Cytometer, the same day.

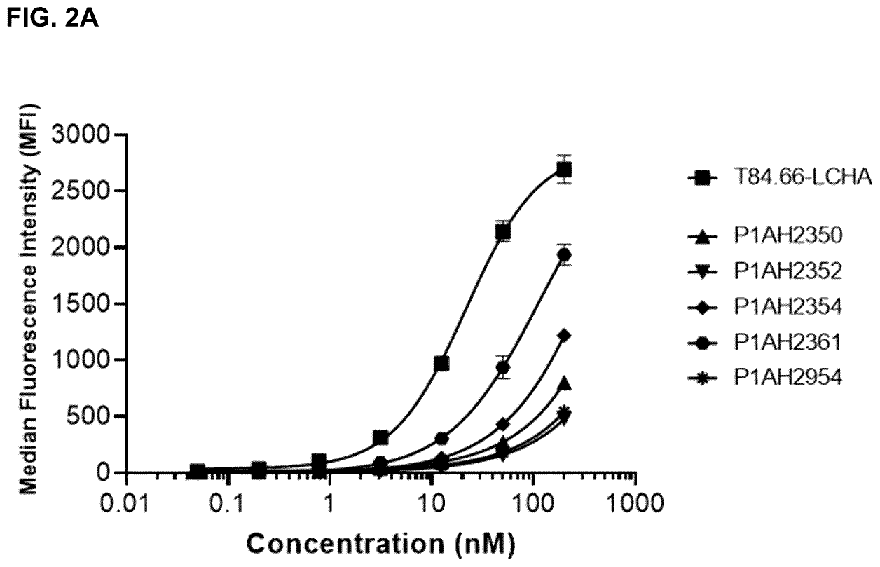

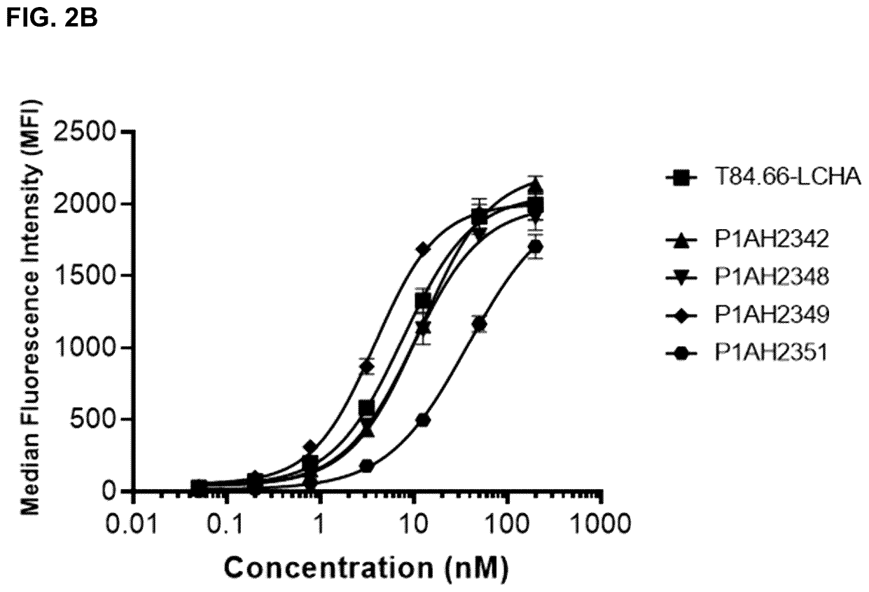

The results are shown in FIG. 2. Variant P1AH2349 was the best variant, showing comparable binding to CEACAM5 and improved expression as compared to the parental binder T84.66-LCHA. This variant was selected for further engineering to reduce MHCII presentation in a second optimization round.

### Example 4—Design and Production of Optimized T84.66 Humanization Variants

In the second optimization round, the best VH variant from round 1, VH5a_Herc-like, was combined with the two most promising VL variants, T84.66-LCHA_VL_127dH and T84.66-LCHA_VL_A51G (variants P1AI5013 and P1AI5014) to assess if a combination of the optimized VH and VL domains would lead to an additional gain in expression titer. The two expression-improving single-point mutations VL-127dH and VL-A51G were combined in an VL variant using the original T84.66-LCHA_VL germline IGKV3-20*02 (T84.66-LCHA_VL_127dH_A51G, used in antibody P1AI5032), and finally in a VL variant based on germline IGKV1-39*01 (VL2a_Herc-like_127dH_A51G, used in antibody P1AI5073). The antibodies that were designed are listed in Table 4.

Furthermore, MHC-associated peptide proteomics (MAPPs) experiments had revealed two MHCII-presented peptides originating from the VL region of T84.66-LCHA_VL: QAPRLLIYRASNRAT (part of VL-framework 2 and LCDR2) and EPEDFAVYYCQQTNEDPYT (part of VL-framework 3 and LCDR3).

In order to reduce the propensity for MHCII presentation, variants of these regions were proposed that are showing a reduced number of MHCII-binding peptides in the NetMHCIIpan 4.0 in silico predictor (considering nine common HLA-DRB1 alleles). The predictions for these variants are given in Table 5. All VL variants with improved in silico MHCII binding are based on the expression-optimized VL sequence T84.66-LCHA_VL_A51G and were combined with the expression-optimized VH domain VH5a_Herc-like (compare Table 4).

TABLE 4
T84.66 humanization variants that were produced in the second optimization round.

| ID | Name of VH | Name of VL | Description |
|---|---|---|---|
| P1AH2338 | T84.66-LCHA_VH | T84.66-LCHA_VL | Reference |
| P1AH2349 | VH5a_Herc-like | T84.66-LCHA_VL | Rehumanized improved expression |
| | | | variant (VH only, from round 1) |
| P1AI5013 | VH5a_Herc-like | T84.66- | Rehumanized improved expression |
| | | LCHA_VL_I27dH | variant |
| P1AI5014 | VH5a_Herc-like | T84.66- | Rehumanized improved expression |
| | | LCHA_VL_A51G | variant |
| P1AI5015 | VH5a_Herc-like | 182_N92Y_D94Y | LCDR3 variant based on T84.66- |
| | | | LCHA_VL_A51G |
| P1AI5016 | VH5a_Herc-like | 362_N92V_D94Y | LCDR3 variant based on T84.66- |
| | | | LCHA_VL_A51G |
| P1AI5017 | VH5a_Herc-like | 722_N92S_D94Y | LCDR3 variant based on T84.66- |
| | | | LCHA_VL_A51G |
| P1AI5018 | VH5a_Herc-like | 902_N92T_D94Y | LCDR3 variant based on T84.66- |
| | | | LCHA_VL_A51G |
| P1AI5019 | VH5a_Herc-like | 4861_Q89G_P95Y | LCDR3 variant based on T84.66- |
| | | | LCHA_VL_A51G |
| P1AI5020 | VH5a_Herc-like | 4862_Q89G_D94Y | LCDR3 variant based on T84.66- |
| | | | LCHA_VL_A51G |
| P1AI5021 | VH5a_Herc-like | 4864_Q89G_D94P | LCDR3 variant based on T84.66- |
| | | | LCHA_VL_A51G |
| P1AI5022 | VH5a_Herc-like | 4874_Q89G_D94E | LCDR3 variant based on T84.66- |
| | | | LCHA_VL_A51G |
| P1AI5023 | VH5a_Herc-like | 4876_Q89G_D94H | LCDR3 variant based on T84.66- |
| | | | LCHA_VL_A51G |
| P1AI5024 | VH5a_Herc-like | 5040_Q89G_N92Y | LCDR3 variant based on T84.66- |
| | | | LCHA_VL_A51G |
| P1AI5025 | VH5a_Herc-like | 5580_Q89G_N92S | LCDR3 variant based on T84.66- |
| | | | LCHA_VL_A51G |
| P1AI5026 | VH5a_Herc-like | 5760_Q89G_N92T | LCDR3 variant based on T84.66- |
| | | | LCHA_VL_A51G |
| P1AI5027 | VH5a_Herc-like | 5940_Q89G_N92Q | LCDR3 variant based on T84.66- |
| | | | LCHA_VL_A51G |
| P1AI5028 | VH5a_Herc-like | 6120_Q89G_N92H | LCDR3 variant based on T84.66- |
| | | | LCHA_VL_A51G |
| P1AI5029 | VH5a_Herc-like | 6300_Q89G_N92K | LCDR3 variant based on T84.66- |
| | | | LCHA_VL_A51G |
| P1AI5030 | VH5a_Herc-like | 11340_V85Y_Q89G | LCDR3 variant based on T84.66- |
| | | | LCHA_VL_A51G |
| P1AI5031 | VH5a_Herc-like | 24_A55M | LCDR2 variant based on T84.66- |
| | | | LCHA_VL_A51G |
| P1AI5032 | VH5a_Herc-like | T84.66- | Rehumanized improved expression |
| | | LCHA_VL_I27dH_A51G | variant (double mutant) |
| P1AI5073 | VH5a_Herc-like | VL2a_Herc- | Rehumanized improved expression |
| | | like_I27dH_A51G | variant (double mutant) with |
| | | | alternative VL framework |

TABLE 5
Predicted number of strong and weak MHCII binding peptides
in the designed VL sequences (NetMHCIIpan 4.0). Predictor
settings: Strong threshold 1, weak threshold 5, alleles
DRB1_0101, DRB1_0301, DRB1_0401, DRB1_0701, DRB1_0801,
DRB1_0901, DRB1_1101, DRB1_1301, DRB1_1501.
Peptides occurring in at least 10 human V region
germline sequences were not counted.

| Sequence | n_strong | n_weak | n_total |
|---|---|---|---|
| T84.66-LCHA_VL | 23 | 33 | 56 |
| T84.66-LCHA_VL_A51G | 17 | 36 | 53 |
| 182_N92Y_D94Y | 14 | 32 | 46 |
| 362_N92V_D94Y | 14 | 32 | 46 |
| 722_N92S_D94Y | 14 | 32 | 46 |
| 902_N92T_D94Y | 14 | 32 | 46 |
| 4861_Q89G_P95Y | 14 | 32 | 46 |
| 4862_Q89G_D94Y | 14 | 32 | 46 |
| 4864_Q89G_D94P | 15 | 31 | 46 |
| 4874_Q89G_D94E | 15 | 31 | 46 |
| 4876_Q89G_D94H | 15 | 31 | 46 |
| 5040_Q89G_N92Y | 15 | 31 | 46 |
| 5580_Q89G_N92S | 15 | 31 | 46 |
| 5760_Q89G_N92T | 15 | 31 | 46 |
| 5940_Q89G_N92Q | 15 | 31 | 46 |
| 6120_Q89G_N92H | 15 | 31 | 46 |
| 6300_Q89G_N92K | 15 | 31 | 46 |
| 11340_V85Y_Q89G | 15 | 31 | 46 |
| 24_A55M | 12 | 36 | 48 |

The predicted number of MHCII binding peptides, especially strong ones, in the VL of the optimized T84.66 humanization variants is reduced as compared to T84.66-LCHA_VL.

The T84.66 humanization variants combining the optimized VH and VL (variants P1AI5013 and P1AI5014) and designed to reduce MHCII presentation were produced in human IgG₁ format as described in Example 2. Table 6 summarizes the outcome of the production.

TABLE 6
Yield and composition as main peak CE-SDS of the purified T84.66
humanization variants from the second optimization round.

| | prepSEC yield | CE-SDS monomer |
|---|---|---|
| Variant | [μg] | peak [%] |
| P1AH2338 (T84.66-LCHA) | 445.9 | 98.7 |
| P1AH2349 (Rehumanized | 1816.6 | 98.0 |
| improved expression variant) | | |
| P1AI5013 (Rehumanized | 2119.5 | 98.3 |
| improved expression variant) | | |
| P1AI5014 (Rehumanized | 2289.7 | 98.3 |
| improved expression variant) | | |
| P1AI5015 | 2225.4 | 98.1 |
| P1AI5016 | 2846.3 | 98.3 |
| P1AI5017 | 2395.0 | 98.1 |
| P1AI5018 | 2667.6 | 98.4 |
| P1AI5019 | 3437.4 | 98.9 |
| P1AI5020 | 3540.4 | 98.5 |
| P1AI5021 | 2874.1 | 98.4 |
| P1AI5022 | 3984.3 | 98.5 |
| P1AI5023 | 2390.0 | 98.4 |
| P1AI5024 | 2731.0 | 98.4 |
| P1AI5025 | 3013.7 | 98.4 |
| P1AI5026 | 2611.8 | 98.4 |
| P1AI5027 | 3150.5 | 98.1 |
| P1AI5028 | 3156.3 | 98.5 |
| P1AI5029 | 4210.4 | 98.5 |
| P1AI5030 | 3483.6 | 98.4 |
| P1AI5031 | 2103.6 | 98.4 |
| P1AI5032 | 2164.4 | 98.3 |
| P1AI5073 | 2073.1 | 98.1 |

The combination of the optimized VH and VL (antibodies P1AI5013 and P1AI5014) led to a slight increase in yield compared to the rehumanized improved expression variant P1AH2349.

Introduction of mutations to reduce the number of MHCII-binding peptides led to variants with a good yield (up to 2 fold increase compared to P1AH2349).

### Example 5—Binding of the Optimized T84.66 Humanization Variants to Cells

To determine if the binding to CEACAM5 was impacted by the mutations introduced during the second round of optimization, binding of the optimized T84.66 humanization variants from Example 4 to human CEACAM5 expressing cells was assessed by FACS and EC50 calculated, as described in Example 3.

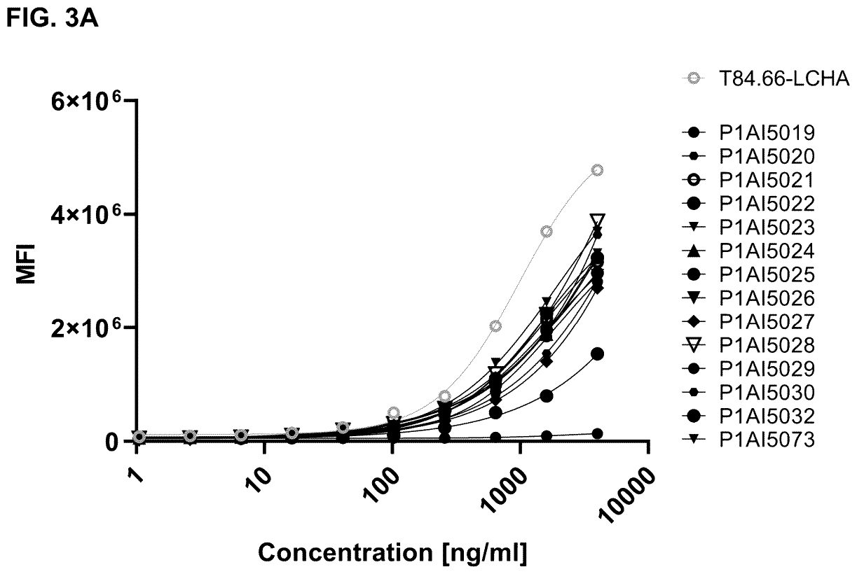

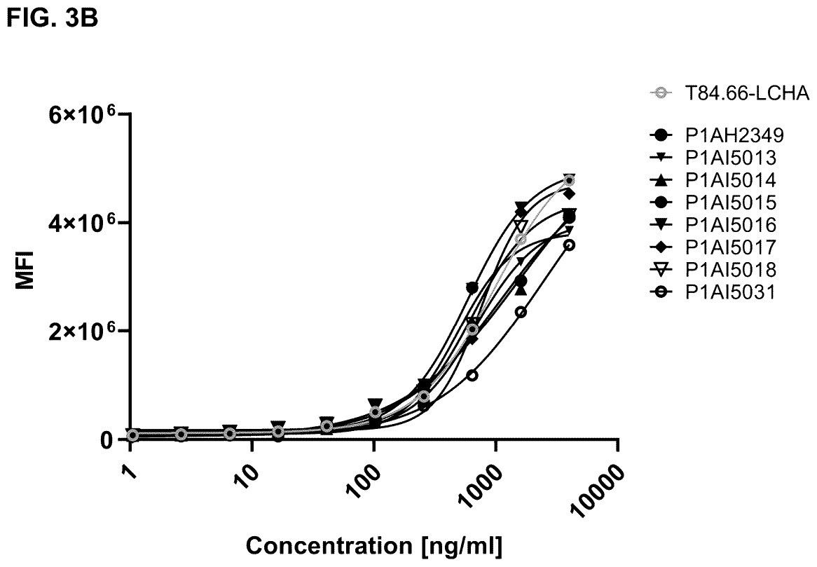

Results are shown in FIG. 3 and Table 7.

TABLE 7
EC50 from FACS binding study with
MKN-45 CEACAM5 expressing cells.

| | EC50 |
|---|---|
| Variant | [ng/ml] |
| P1AH2349 | 1109.0 |
| P1AI5013 | 684.7 |
| P1AI5014 | 1535.0 |
| P1AI5015 | 483.4 |
| P1AI5016 | 587.9 |
| P1AI5017 | 761.7 |
| P1AI5018 | 650.5 |
| P1AI5019 | 2487.0 |
| P1AI5020 | na |
| P1AI5021 | 1622.0 |
| P1AI5022 | na |
| P1AI5023 | 7206.0 |
| P1AI5024 | 11461.0 |
| P1AI5025 | 2444.0 |
| P1AI5026 | 1829.0 |
| P1AI5027 | 25109.0 |
| P1AI5028 | 7512.0 |
| P1AI5029 | 1918.0 |
| P1AI5030 | 11154.0 |
| P1AI5031 | 2415.0 |
| P1AI5032 | 1534.0 |
| P1AI5073 | 1908.0 |
| T84.66-LCHA | 988.7 |

The optimized T84.66 humanization variants showing a similar binding to the target as the parental T84.66-LCHA binder (P1AI5014, P1AI5015, P1AI5016, P1AI5017, P1AI5018 and P1AI5031) were further characterized for binding to human CEACAM5, CEACAM1 and CEACAM6. The assays were performed as described in the following.

MKN-45 (DSMZ ACC409) cells were collected using trypsin and viability was checked. Then 100 μl of re-suspended cells were seeded into a V-bottom plate (200′000 cells/well). The antibodies were diluted in FACS buffer. The plates were centrifuged for 4 min at 400×g and supernatant was removed. Then 25 μl/well of the diluted antibodies were added to the cells. The cells were incubated for 45 min at 4° C. After the incubation, cells were washed twice and 25 μl/well of diluted (1/100 in FACS buffer) PE anti-human Fc secondary antibody (Jackson Immuno Research, #109-116-170) was added to the cells. The cells were incubated for 45 min at 4° C. After the incubation, cells were washed twice. Then cells were fixed with 1% PFA at 4° C. for 20 min. After incubation cells were washed twice and re-suspended in 125 pl/well of FACS buffer. Cell binding was measured on the BD LSRFortessa Flow Cytometer.

Viability of CHO-CEACAM1 (CHO cells overexpressing human CEACAM1) and CHO-CEACAM6 (CHO cells overexpressing human CEACAM6) cells was checked. Then 100 μl of re-suspended target cells were seeded into a U-bottom plate (100′000 cells/well). The antibodies were diluted in FACS buffer. The plates were centrifuged for 4 min at 400×g and supernatant was removed. Then 25 pl/well of the diluted antibodies were added to the cells. The cells were incubated for 45 min at 4° C. After the incubation, cells were washed twice and 25 μl/well of diluted (1/100 in FACS buffer) PE anti-human Fc secondary antibody (Jackson Immuno Research, #109-116-170) or the directly labeled CD66 antibody (Clone B1.1/CD66, BD Bioscience, #551480) was added to the cells. The cells were incubated for 45 min at 4° C. After the incubation, cells were washed twice. Then cells were re-suspended in 150 μl/well of FACS buffer. Cell binding was measured on the BD LSRFortessa Flow Cytometer, the same day.

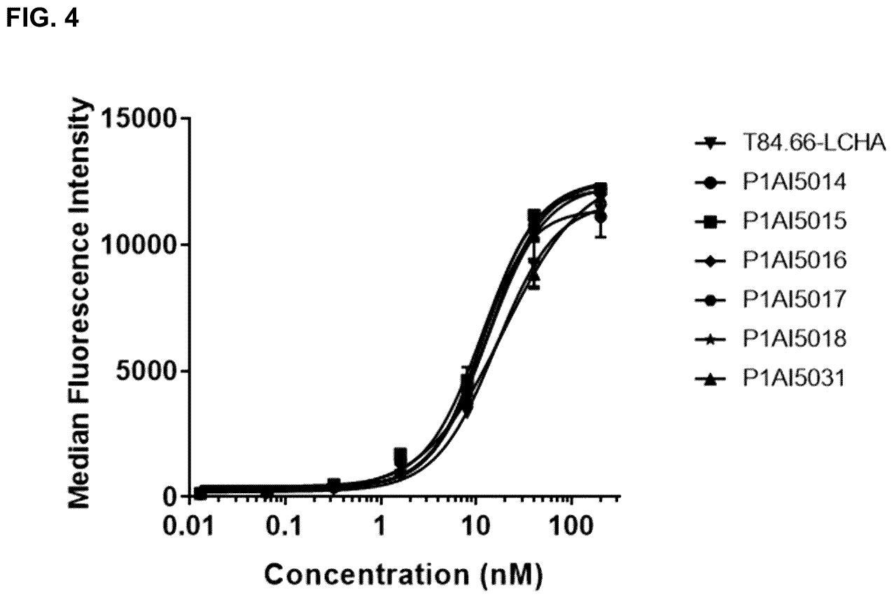

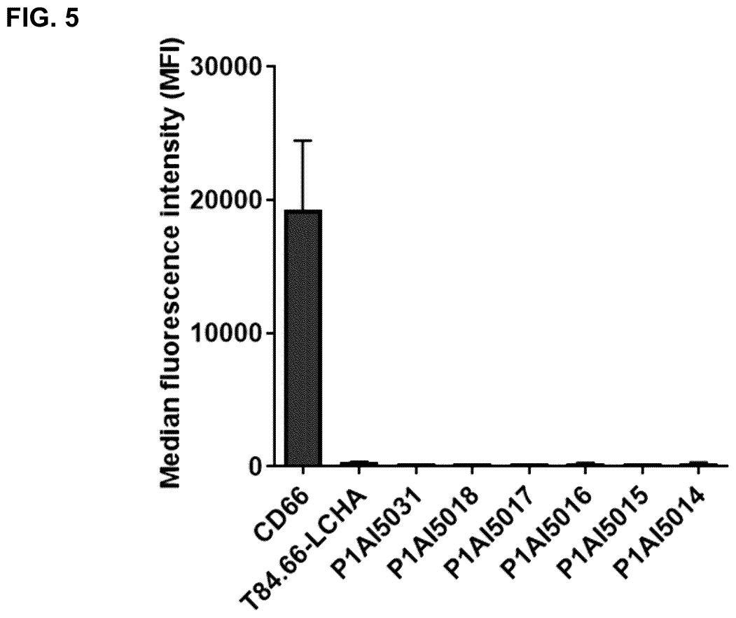

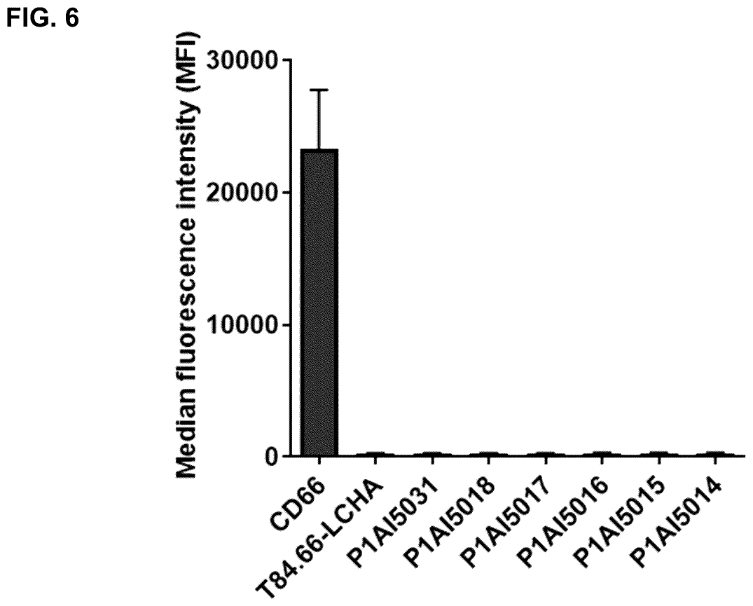

Results are shown in FIG. 4 to FIG. 6.

The selected optimized T84.66 humanization variants show a similar binding to human CEACAM5 as the parental clone T84.66-LCHA, they do not bind to human CEACAM1 and 6, and they have a reduced number of MHCII-binding peptides in silico predicted by NetMHCIIpan 4.0.

The selected optimized T84.66 humanization variants did not lose binding to the target after incubation for two weeks at 37° C. and had aggregation temperatures and hydrophobicity scores similar to the parental binders and to other IgGs (data not shown).

### Example 6—Functional Activity of Optimized T84.66 Humanization Variants as T-Cell Bispecific Antibodies

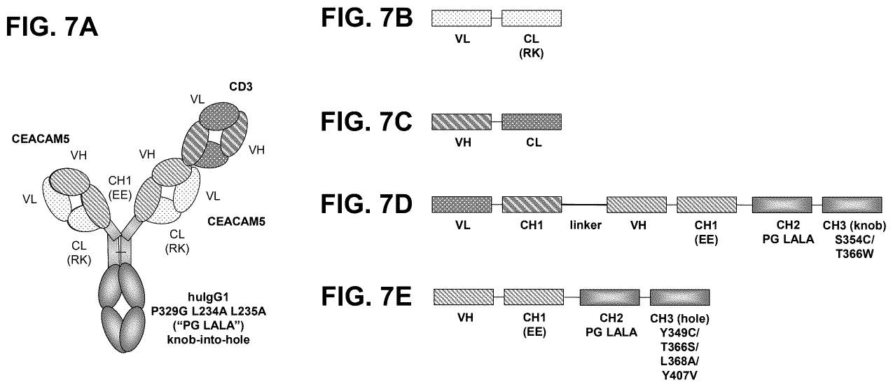

Selected optimized T84.66 humanization variants were produced as T-cell bispecific antibodies (“TCBs”), in a “2+1” format monovalently binding to CD3 (CD3 binder of SEQ ID NOs 45-52) and bivalently binding to CEACAM5. A schematic illustration of the TCBs used in this Example in provided in FIG. 7.

Functional activity of the TCB containing the parental CEACAM5 binder T84.66-LCHA and of the corresponding TCB containing the optimized CEACAM5 binder P1AI50131 as exemplary binder of the invention was tested in a Jurkat NFAT reporter cell assay on CEACAM5 expressing LS180 cells.

In this assay, upon simultaneous binding of CEACAM5-TCB to CEACAM5 positive target cells and CD3 antigen (expressed on Jurkat-NFAT reporter cells), the NFAT promoter is activated and leads to expression of active firefly luciferase. The intensity of luminescence signal (obtained upon addition of the luciferase substrate) is proportional to the intensity of CD3 activation and signaling and can be measured as an activation marker.

Jurkat NFAT reporter cells (GloResponse Jurkat NFAT-RE-luc2P; Promega #CS176501) are a human acute lymphatic leukemia reporter cell line with a NFAT promoter, expressing human CD3. The cells were cultured in advanced RPMI 1640, 2% FCS, 1% Glutamax at 0.1-0.5 mio cells per ml. A final concentration of 200 μg per ml hygromycin B was added whenever cells were passaged. Cells were harvested and viability was checked. LS180 (ATCC CL-187) cells were collected using trypsin and viability was checked. Then 20 μl of a mix of LS180 (10′000 cells/well) and Jurkat NFAT (20′000 cells/well) cells were seeded into a flat-bottom, white-walled 384-well-plate (Falcon Corning #353988) and 20 μl of the diluted antibodies or medium were added. After 5 h, 40 μl/well of the reconstituted ONE-Glo Substrate (Promega #E6210) were added and after 3 min plate luminescence was measured on a Tecan Spark 10 M reader (luminescence 0.5 sec).

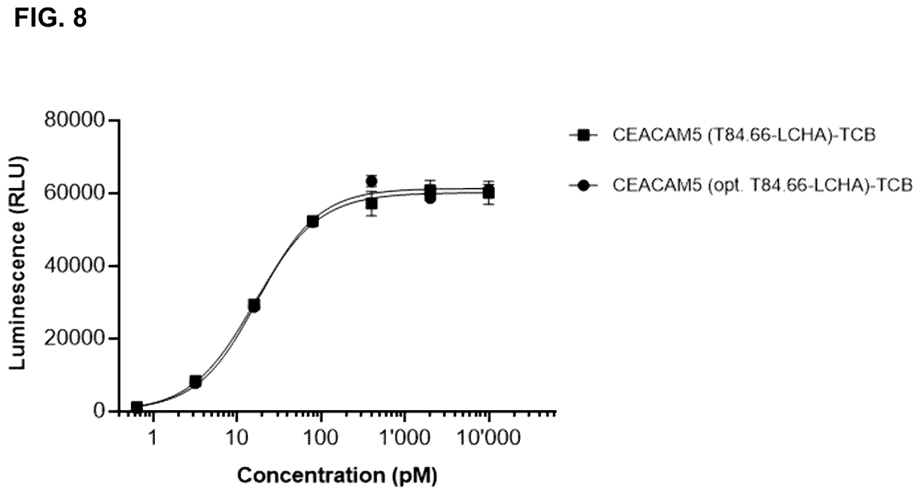

Both tested TCBs show a comparable dose dependent activation of Jurkat NFAT cells upon crosslinking on CEACAM5 positive LS180 cells (FIG. 8).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-25. (canceled)

26. An antibody that binds to CEACAM5, comprising:
a heavy chain variable region (VH_{CEACAM5}) comprising a heavy chain complementarity determining region (HCDR) 1 of SEQ ID NO: 1, an HCDR 2 of SEQ ID NO: 12, and an HCDR 3 of SEQ ID NO: 3; and
a light chain variable region (VL_{CEACAM5}) comprising a light chain complementarity determining region (LCDR) 1 of SEQ ID NO: 5.

27. The antibody of claim 26, wherein the antibody comprises an Fc region.

28. The antibody of claim 27, wherein the Fc region is an IgG Fc region.

29. The antibody of claim 28, wherein the Fc region is a human IgG₁ Fc region.

30. The antibody of claim 27, wherein the antibody is a full-length antibody.

31. The antibody of claim 30, wherein the antibody is an IgG antibody.

32. The antibody of claim 31, wherein the antibody is an IgG₁ antibody.

33. A fragment of the antibody of claim 26, wherein the antibody is selected from the group consisting of an Fv molecule, a scFv molecule, a Fab molecule, and a F(ab′)₂ molecule.

34. The antibody of claim 26, wherein the antibody is a multispecific antibody.

35. The antibody of claim 34, wherein the multispecific antibody is a bispecific antibody.

36. The antibody of claim 35, wherein the bispecific antibody specifically binds to CD3.

37. An isolated polynucleotide encoding an antibody that binds to CEACAM5 comprising:
a heavy chain variable region (VH_{CEACAM5}) comprising a heavy chain complementarity determining region (HCDR) 1 of SEQ ID NO: 1, an HCDR 2 of SEQ ID NO: 12, and an HCDR 3 of SEQ ID NO: 3; and
a light chain variable region (VL_{CEACAM5}) comprising a light chain complementarity determining region (LCDR) 1 of SEQ ID NO: 5.

38. A host cell comprising the isolated polynucleotide of claim 37.

39. A method of producing an antibody that binds to CEACAM5, comprising:
culturing a host cell comprising an isolated polynucleotide encoding the antibody that comprises
a heavy chain variable region (VH_{CEACAM5}) comprising a heavy chain complementarity determining region (HCDR) 1 of SEQ ID NO: 1, an HCDR 2 of SEQ ID NO: 12, and an HCDR 3 of SEQ ID NO: 3; and
a light chain variable region (VL_{CEACAM5}) comprising a light chain complementarity determining region (LCDR) 1 of SEQ ID NO: 5,
and recovering the antibody.

40. An antibody produced by the method of claim 39.

41. A pharmaceutical composition comprising the antibody of claim 26 or produced by the method of claim 39 and a pharmaceutically acceptable carrier.

42. A method of treating an individual in need thereof comprising:
administering to the individual an effective amount of an antibody that binds to CEACAM5, comprising
a heavy chain variable region (VH_{CEACAM5}) comprising a heavy chain complementarity determining region (HCDR) 1 of SEQ ID NO: 1, an HCDR 2 of SEQ ID NO: 12, and an HCDR 3 of SEQ ID NO: 3; and
a light chain variable region (VL_{CEACAM5}) comprising a light chain complementarity determining region (LCDR) 1 of SEQ ID NO: 5,
or a pharmaceutical composition comprising the antibody.

43. The method of treating an individual of claim 42, wherein the individual has cancer.

44. An antibody that binds to CEACAM5, comprising a heavy chain variable region (VH_{CEACAM5}) comprising an amino acid sequence that is at least about 95%, 96%, 97%, 98%, 99% or 100% identical to the amino acid sequence of SEQ ID NO: 13.

45. An antibody that binds to CEACAM5, comprising:
a heavy chain variable region (VH_{CEACAM5}) comprising a heavy chain complementarity determining region (HCDR) 1 of SEQ ID NO: 1, an HCDR 2 of SEQ ID NO: 12, and an HCDR 3 of SEQ ID NO: 3; and
a light chain variable region (VL_{CEACAM5}) comprising a light chain complementarity determining region (LCDR) 1 of SEQ ID NO: 5 and at least one set selected from the group consisting of:
(1) an LCDR 2 of SEQ ID NO: 26 and an LCDR 3 of SEQ ID NO: 28;
(2) an LCDR 2 of SEQ ID NO: 14 and an LCDR 3 of SEQ ID NO: 20;
(3) an LCDR 2 of SEQ ID NO: 14 and an LCDR 3 of SEQ ID NO: 16;
(4) an LCDR 2 of SEQ ID NO: 14 and an LCDR 3 of SEQ ID NO: 18;
(5) an LCDR 2 of SEQ ID NO: 14 and an LCDR 3 of SEQ ID NO: 22;
(6) an LCDR 2 of SEQ ID NO: 14 and an LCDR 3 of SEQ ID NO: 7;
(7) an LCDR 2 of SEQ ID NO: 24 and an LCDR 3 of SEQ ID NO: 7; and
(8) an LCDR 2 of SEQ ID NO: 10 and an LCDR 3 of SEQ ID NO: 7.

46. An antibody that binds to CEACAM5, comprising:
a heavy chain variable region (VH_{CEACAM5}) comprising an amino acid sequence that is at least about 95%, 96%, 97%, 98%, 99% or 100% identical to the amino acid sequence of SEQ ID NO: 13, and
a light chain variable region (VL_{CEACAM5}) comprising an amino acid sequence that is at least about 95%, 96%, 97%, 98%, 99% or 100% identical to the amino acid sequence of:
(1) SEQ ID NO: 29;
(2) SEQ ID NO: 21;
(3) SEQ ID NO: 17;
(4) SEQ ID NO: 19;
(5) SEQ ID NO: 23;
(6) SEQ ID NO: 15;
(7) SEQ ID NO: 25; or
(8) SEQ ID NO: 11.
